(19) United States
(12) Patent Application Publication
(10) Pub. No.: US 2009/0088441 A1
(43) Pub. Date: Apr. 2, 2009

(54) Quinoline Compounds

(76) Inventors: Leifeng Cheng, Molndal (SE); Michael Karle, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 12/238,982

(22) Filed: Sep. 26, 2008

Related U.S. Application Data
(60) Provisional application No. 60/975,536, filed on Sep. 27, 2007.

(51) Int. Cl. A61K 31/496; C07D 215/14; C07D 401/10; A61K 31/47

(52) U.S. Cl. 514/253.06; 546/168; 514/311; 544/363

(57) ABSTRACT

The present invention relates to novel compounds of the general formula (I)

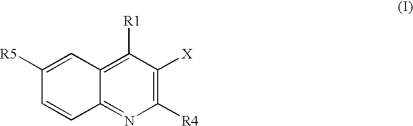

wherein R¹, R⁴, R⁵ and X are as defined,
having a positive allosteric GABA_{B} receptor (GBR) modulator effect, methods for the preparation of said compounds and to their use, optionally in combination with a GABA_{B} agonist, for the inhibition of transient lower esophageal sphincter relaxations, for the treatment of gastroesophageal reflux disease, as well as for the treatment of functional gastrointestinal disorders and irritable bowel syndrome (IBS).

### FIELD OF THE INVENTION

The present invention relates to novel compounds having a positive allosteric GABA_{B} receptor (GBR) modulator effect, methods for the preparation of said compounds and their use for the inhibition of transient lower esophageal sphincter relaxations, for the treatment of gastroesophageal reflux disease, as well as for the treatment of functional gastrointestinal disorders and irritable bowel syndrome (IBS).

### BACKGROUND OF THE INVENTION

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastroesophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, recent research (e.g. Holloway & Dent (1990) Gastroenterol. Clin. N. Amer. 19, pp. 517-535) has shown that most reflux episodes occur during transient lower esophageal sphincter relaxations (TLESR), i.e. relaxations not triggered by swallows. It has also been shown that gastric acid secretion usually is normal in patients with GERD.

Consequently, there is a need for a therapy that reduces the incidence of TLESR and thereby prevents reflux.

GABA_{B}-receptor agonists have been shown to inhibit TLESR, which is disclosed in WO 98/11885 A1.

Functional gastrointestinal disorders, such as functional dyspepsia, can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II. Functional Gastrointestinal Disorders. Diagnosis, Pathophysiology and Treatment. 2 ed. McLean, Va. Degnon Associates, Inc.; 2000.351-432 and Drossman D A, Corazziari E, Talley N J, Thompson W G and Whitehead W E. Rome II: A multinational consensus document on Functional Gastrointestinal Disorders. Gut 45 (Suppl. 2), II1-II81.9-1-1999.

Irritable bowel syndrome (IBS) can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders. Diagnosis, Pathophysiology and Treatment. 2 ed. McLean, Va. Degnon Associates, Inc.; 2000.351-432 and Drossman D A, Corazziari E, Talley N J, Thompson W G and Whitehead W E. Rome II: A multinational consensus document on Functional Gastrointestinal Disorders. Gut 45 (Suppl. 2), II1-II81.9-1-1999.

### GABA_{B} Receptor Agonists

GABA (4-aminobutanoic acid) is an endogenous neurotransmitter in the central and peripheral nervous systems. Receptors for GABA have traditionally been divided into GABA_{A} and GABA_{B} receptor subtypes. GABA_{B} receptors belong to the superfamily of G-protein coupled receptors (GPCRs).

The most studied GABA_{B} receptor agonist baclofen (4-amino-3-(p-chlorophenyl)butanoic acid; disclosed in CH 449046) is useful as an antispastic agent. EP 356128 A2 describes the use of the GABA_{B} receptor agonist (3-aminopropyl)methylphosphinic acid for use in therapy, in particular in the treatment of central nervous system disorders.

EP 463969 A1 and FR 2722192 A1 disclose 4-aminobutanoic acid derivatives having different heterocyclic substituents at the 3-carbon of the butyl chain. EP 181833 A1 discloses substituted 3-aminopropylphosphinic acids having high affinities towards GABA_{B} receptor sites. EP 399949 A1 discloses derivatives of (3-aminopropyl)methylphosphinic acid, which are described as potent GABA_{B} receptor agonists. Still other (3-aminopropyl)methylphosphinic acids and (3-aminopropyl)phosphinic acids have been disclosed in WO 01/41743 A1 and WO 01/42252 A1, respectively. Structure-activity relationships of several phosphinic acid analogues with respect to their affinities to the GABA_{B} receptor are discussed in J. Med. Chem. (1995), 38, 3297-3312. Sulphinic acid analogues and their GABA_{B} receptor activities are described in Bioorg. & Med. Chem. Lett. (1998), 8, 3059-3064. For a more general review on GABA_{B} ligands, see Curr. Med. Chem.-Central Nervous System Agents (2001), 1, 27-42.

### Positive Allosteric Modulation of GABA_{B} Receptors

2,6-Di-tert-butyl-4-(3-hydroxy-2,2-dimethylpropyl)phenol (CGP7930) and 3-(3,5-di-tert-butyl-4-hydroxyphenyl)-2,2-dimethylpropanal (disclosed in U.S. Pat. No. 5,304,685) have been described to exert positive allosteric modulation of native and recombinant GABA_{B} receptor activity (Society for Neuroscience, 30^{th} Annual Meeting, New Orleans, La., Nov. 4-9, 2000: Positive Allosteric Modulation of Native and Recombinant GABA_{B} Receptor Activity, S. Urwyler et al.; Molecular Pharmacol. (2001), 60, 963-971).

N,N-Dicyclopentyl-2-methylsulfanyl-5-nitro-pyrimidine-4,6-diamine has been described to exert positive allosteric modulation of the GABA_{B} receptor (The Journal of Pharmacology and Experimental Therapeutics, 307 (2003), 322-330).

### Quinoline Derivatives

Chemistry Letters, 2005, 34(3), 314-315, discloses a Friedlaender synthesis of quinolines by using SnCl₂-2H₂O.

US20060094754 A1 (Hoffmann La Roche) discloses the preparation of quinolines as allosteric enhancers of the GABA_{B} receptors.

US20050080105 A1 (Bristol-Myers Squibb Company, USA) discloses the preparation of 3-thia-4-arylquinolin-2-ones for treating conditions affected by abnormal potassium channel activity.

JP2005060247 (Takeda) discloses the preparation of isoquinolines and their use as selective c-Jun N-terminal kinase (JNK) inhibitors and (pro)drugs.

WO 2004/014860 A2 (Takeda) discloses fused heterocyclic compounds as having peptidase-inhibitory activity being useful as a prophylactic or therapeutic agent against diabetes and the like.

### Outline of the Invention

The present invention provides a compound of the formula

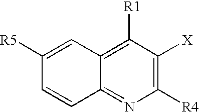

wherein
X is —CO—R⁶ or a group CH(R³)—R²
R¹ is selected from phenyl substituted by one or more of halogen;
R² is selected from aryloxy substituted by one or more of C₁-C₁₀-alkyl, C₁-C₁₀-alkoxy, hydroxy, halogen, cyano, C₁-C₁₀-alkylsulfonyl, di-C₁-C₁₀-alkylamino, or carbamoyl; heteroaryloxy; heteroaryl substituted by one or more of oxo;
R³ is selected from hydrogen or C₁-C₁₀-alkyl;
R⁴ is selected from C₁-C₁₀-alkyl;
R⁵ is selected from halogen or heterocyclyl unsubstituted or substituted by one or more of C₁-C₁₀-alkyl;
R⁶ is O—C(R⁷)(R⁸)(R⁹), wherein R⁷, R⁸ and R⁹ are each independently C₁-C₁₀-alkyl, provided that R⁶ is C₁-C₁₀-alkoxy; and pharmaceutically acceptable salts thereof.
For R⁶ it is understood that to O—C three C₁-C₁₀-alkyl chains are bound. Furthermore, the lowest number of C₁-C₁₀-alkoxy for R⁶ is C₄-alkoxy.

In another embodiment:

R¹ is selected from 4-fluorophenyl;
R² is selected from phenoxy substituted by one or more of isopropyl, methoxy, hydroxy, chloro, cyano, methanesulfonyl, dimethylamino, or carbamoyl; pyridinyloxy; 2-pyridin-2(1H)-onyl;
R³ is selected from hydrogen or methyl;
R⁴ is selected from methyl;
R⁵ is selected from bromo, 1-piperidinyl or 4-methyl-1-piperazinyl;
R⁶ is selected from tert-butoxy.

In another embodiment, the present invention relates to the compounds as denoted in Examples 2, 4-20, 22, and 25.

The general terms used in the definition of formula (I) have the following meanings:

C₁-C₁₀ alkyl is a straight or branched alkyl group, having from 1 to 10 carbon atoms, for example methyl, ethyl, n-propyl, isopropyl, n-butyl, isobutyl, secondary butyl, tertiary butyl, pentyl, isopentyl, hexyl or heptyl.
C₁-C₁₀ alkoxy is an alkoxy group having 1 to 10 carbon atoms, for example methoxy, ethoxy, n-propoxy, n-butoxy, isopropoxy, isobutoxy, secondary butoxy, tertiary butoxy, pentoxy, hexoxy or a heptoxy group.

The term aryl is herein defined as an aromatic ring having from 6 to 14 carbon atoms including both single rings and polycyclic compounds, such as phenyl, benzyl or naphthyl. Analogously, examples of aryloxy are phenoxy, benzyloxy and naphthyloxy.

The term heteroaryl is herein defined as an aromatic ring having 3 to 14 carbon atoms, including both single rings and polycyclic compounds in which one or several of the ring atoms is either oxygen, nitrogen or sulphur, such as pyrazolyl, benzothiadiazolyl, benzothiazolyl, thienyl, imidazolyl, isoxazolyl, pyridinyl and pyrrolyl. Analogously, examples of heteroaryloxy are pyrazolyloxy, benzothiadiazolyloxy, benzothiazolyloxy, thienyloxy, imidazolyloxy, isoxazolyloxy, pyridinyloxy and pyrrolyloxy.

An example of heteroaryl substituted by one or more of oxo is 2-pyridin-2(1H)-onyl.

Halogen as used herein is selected from chlorine, fluorine, bromine or iodine.

The term heterocyclyl is herein defined as a saturated ring having 3 to 14 carbon atoms, including both single rings and polycyclic compounds in which one or several of the ring atoms is either oxygen, nitrogen or sulphur, such as pyrrolidinyl, piperidinyl, piperazinyl, morpholinyl, and thiomorpholinyl.

When two or more groups are used in connection with each other, it means that each group is substituted by the immediately preceding group. For instance, C₁-C₁₀-alkylsulfonyl means a sulfonyl group substituted by a C₁-C₁₀ alkyl group.

When a group is substituted by two or more further groups, these further groups need not be the same. For instance, in di-C₁-C₁₀-alkylamino, the C₁-C₁₀ alkyl groups may be the same or different C₁-C₁₀ alkyl groups.

When the compounds of formula (I) have at least one asymmetric carbon atom, they can exist in several stereochemical forms. The present invention includes the mixture of isomers as well as the individual stereoisomers. The present invention further includes geometrical isomers, rotational isomers, enantiomers, racemates and diastereomers.

Where applicable, the compounds of formula (I) may be used in neutral form, e.g. as a carboxylic acid, or in the form of a salt, preferably a pharmaceutically acceptable salt such as the sodium, potassium, ammonium, calcium or magnesium salt of the compound at issue.

The compounds of formula (I) are useful as positive allosteric GBR (GABA_{B} receptor) modulators. A positive allosteric modulator of the GABA_{B} receptor is defined as a compound which makes the GABA_{B} receptor more sensitive to GABA and GABA_{B} receptor agonists by binding to the GABA_{B} receptor protein at a site different from that used by the endogenous ligand. The positive allosteric GBR modulator acts synergistically with an agonist and increases potency and/or intrinsic efficacy of the GABA_{B} receptor agonist. It has also been shown that positive allosteric modulators acting at the GABA_{B} receptor can produce an agonistic effect. Therefore, compounds of formula (I) can be effective as full or partial agonists.

The compounds may be used as a positive allosteric GABA_{B} receptor modulator. Also envisaged is a pharmaceutical composition comprising a compound above as an active ingredient and a pharmaceutically acceptable carrier or diluent.

A further aspect of the invention is a compound of the formula (I) above for use in therapy.

As a consequence of the GABA_{B} receptor becoming more sensitive to GABA_{B} receptor agonists upon the administration of a positive allosteric modulator, an increased inhibition of transient lower esophageal sphincter relaxations (TLESR) for a GABA_{B} agonist is observed. Consequently, the present invention is directed to the use of a positive allosteric GABA_{B} receptor modulator according to formula (I), optionally in combination with a GABA_{B} receptor agonist, for the preparation of a medicament for the inhibition of transient lower esophageal sphincter relaxations (TLESRs).

A further aspect of the invention is the use of a compound of formula (I), optionally in combination with a GABA_{B} receptor agonist, for the manufacture of a medicament for the prevention of reflux.

Also envisaged is a compound of formula (I) for use in the treatment of gastroesophageal reflux disease (GERD).

Also envisaged is a compound of formula (I) for use in the prevention of reflux.

Also envisaged is a compound of formula (I) for use in the inhibition of transient lower esophageal sphincter relaxations (TLESRs).

Also envisaged is a compound of formula (I) for use in the treatment of a functional gastrointestinal disorder. The functional gastrointestinal disorder could be e g functional dyspepsia.

Also envisaged is a compound of formula (I) for use in the treatment of irritable bowel syndrome (IBS). Said IBS could be e g constipation predominant IBS, diarrhea predominant IBS, or alternating bowel movement predominant IBS.

Still a further aspect of the invention is the use of a compound of formula (I), optionally in combination with a GABA_{B} receptor agonist, for the manufacture of a medicament for the treatment of gastroesophageal reflux disease (GERD).

Effective management of regurgitation in infants would be an important way of preventing, as well as curing lung disease due to aspiration of regurgitated gastric contents, and for managing failure to thrive, inter alia due to excessive loss of ingested nutrient. Thus, a further aspect of the invention is the use of a compound of formula (I), optionally in combination with a GABA_{B} receptor agonist, for the manufacture of a medicament for the treatment of lung disease.

Another aspect of the invention is the use of a compound of formula (I), optionally in combination with a GABA_{B} receptor agonist, for the manufacture of a medicament for the management of failure to thrive.

Another aspect of the invention is the use of a compound of formula (I), optionally in combination with a GABA_{B} receptor agonist, for the manufacture of a medicament for the treatment or prevention of asthma, such as reflux-related asthma.

A further aspect of the invention is the use of a compound of formula (I), optionally in combination with a GABA_{B} receptor agonist, for the manufacture of a medicament for the treatment or prevention of laryngitis or chronic laryngitis.

A further aspect of the present invention is a method for the inhibition of transient lower esophageal sphincter relaxations (TLESRs), whereby a pharmaceutically and pharmacologically effective amount of a compound of formula (I), optionally in combination with a GABA_{B} receptor agonist, is administered to subject in need of such inhibition.

Another aspect of the invention is a method for the prevention of reflux, whereby a pharmaceutically and pharmacologically effective amount of a compound of formula (I), optionally in combination with a GABA_{B} receptor agonist, is administered to a subject in need of such prevention.

Still a further aspect of the invention is a method for the treatment of gastroesophageal reflux disease (GERD), whereby a pharmaceutically and pharmacologically effective amount of a compound of formula (I), optionally in combination with a GABA_{B} receptor agonist, is administered to a subject in need of such treatment.

Another aspect of the present invention is a method for the treatment or prevention of regurgitation, whereby a pharmaceutically and pharmacologically effective amount of a compound of formula (I), optionally in combination with a GABA_{B} receptor agonist, is administered to a subject in need of such treatment.

Yet another aspect of the invention is a method for the treatment or prevention of regurgitation in infants, whereby a pharmaceutically and pharmacologically effective amount of a compound of formula (I), optionally in combination with a GABA_{B} receptor agonist, is administered to a subject in need of such treatment.

Still a further aspect of the invention is a method for the treatment, prevention or inhibition of lung disease, whereby a pharmaceutically and pharmacologically effective amount of a compound of formula (I), optionally in combination with a GABA_{B} receptor agonist, is administered to a subject in need of such treatment. The lung disease to be treated may inter alia be due to aspiration of regurgitated gastric contents.

Still a further aspect of the invention is a method for the management of failure to thrive, whereby a pharmaceutically and pharmacologically effective amount of a compound of formula (I), optionally in combination with a GABA_{B} receptor agonist, is administered to a subject in need of such treatment.

A further aspect of the invention is a method for the treatment or prevention of asthma, such as reflux-related asthma, whereby a pharmaceutically and pharmacologically effective amount of a compound of formula (I), optionally in combination with a GABA_{B} receptor agonist, is administered to a subject in need of such treatment.

A further aspect of the invention is a method for the treatment or prevention of laryngitis or chronic laryngitis, whereby a pharmaceutically and pharmacologically effective amount of a compound of formula (I), optionally in combination with a GABA_{B} receptor agonist, is administered to a subject in need of such treatment.

A further embodiment is the use of a compound of formula (I), optionally in combination with a GABA_{B} receptor agonist, for the manufacture of a medicament for the treatment of a functional gastrointestinal disorder (FGD). Another aspect of the invention is a method for the treatment of a functional gastrointestinal disorder, whereby an effective amount of a compound of formula (I), optionally in combination with a GABA_{B} receptor agonist, is administered to a subject suffering from said condition.

A further embodiment is the use of a compound of formula (I), optionally in combination with a GABA_{B} receptor agonist, for the manufacture of a medicament for the treatment of functional dyspepsia. Another aspect of the invention is a method for the treatment of functional dyspepsia, whereby an effective amount of a compound of formula (I), optionally in combination with a GABA_{B} receptor agonist, is administered to a subject suffering from said condition.

Functional dyspepsia refers to pain or discomfort centered in the upper abdomen. Discomfort may be characterized by or combined with upper abdominal fullness, early satiety, bloating or nausea. Etiologically, patients with functional dyspepsia can be divided into two groups:
1-Those with an identifiable pathophysiological or microbiologic abnormality of uncertain clinical relevance (e.g. Helicobacter pylori gastritis, histological duodenitis, gallstones, visceral hypersensitivity, gastroduodenal dysmotility)
2-Patients with no identifiable explanation for the symptoms.

Functional dyspepsia can be diagnosed according to the following:

At least 12 weeks, which need not be consecutive within the preceding 12 months of
1-Persistent or recurrent dyspepsia (pain or discomfort centered in the upper abdomen) and
2-No evidence of organic disease (including at upper endoscopy) that is likely to explain the symptoms and
3-No evidence that dyspepsia is exclusively relieved by defecation or associated with the onset of a change in stool frequency or form.

Functional dyspepsia can be divided into subsets based on distinctive symptom patterns, such as ulcer-like dyspepsia, dysmotility-like dyspepsia and unspecified (non-specific) dyspepsia.

Currently existing therapy of functional dyspepsia is largely empirical and directed towards relief of prominent symptoms. The most commonly used therapies still include antidepressants.

A further aspect of the invention is the use of a compound according to formula (I), optionally in combination with a GABA_{B} receptor agonist, for the manufacture of a medicament for the treatment or prevention of irritable bowel syndrome (IBS), such as constipation predominant IBS, diarrhea predominant IBS or alternating bowel movement predominant IBS.

A further aspect of the invention is a method for the treatment or prevention of irritable bowel syndrome (IBS), whereby a pharmaceutically and pharmacologically effective amount of a compound of formula (I), optionally in combination with a GABA_{B} receptor agonist, is administered to a subject in need of such treatment.

IBS is herein defined as a chronic functional disorder with specific symptoms that include continuous or recurrent abdominal pain and discomfort accompanied by altered bowel function, often with abdominal bloating and abdominal distension. It is generally divided into 3 subgroups according to the predominant bowel pattern:
1-diarrhea predominant
2-constipation predominant
3-alternating bowel movements.

Abdominal pain or discomfort is the hallmark of IBS and is present in the three subgroups.

IBS symptoms have been categorized according to the Rome criteria and subsequently modified to the Rome II criteria. This conformity in describing the symptoms of IBS has helped to achieve consensus in designing and evaluating IBS clinical studies.

The Rome II diagnostic criteria are:
1-Presence of abdominal pain or discomfort for at least 12 weeks (not necessarily consecutively) out of the preceding year
2-Two or more of the following symptoms:
a) Relief with defecation
b) Onset associated with change in stool frequency
c) Onset associated with change in stool consistency

A further aspect of the invention is the use of a compound according to formula (I), optionally in combination with a GABA_{B} receptor agonist, for the manufacture of a medicament for the treatment or prevention CNS disorders, such as anxiety.

A further aspect of the invention is a method for the treatment or prevention of CNS disorders, such as anxiety, whereby a pharmaceutically and pharmacologically effective amount of a compound of formula (I), optionally in combination with a GABA_{B} receptor agonist, is administered to a subject in need of such treatment.

A further aspect of the invention is the use of a compound according to formula (I), optionally in combination with a GABA_{B} receptor agonist, for the manufacture of a medicament for the treatment or prevention of depression.

A further aspect of the invention is a method for the treatment or prevention of depression, whereby a pharmaceutically and pharmacologically effective amount of a compound of formula (I), optionally in combination with a GABA_{B} receptor agonist, is administered to a subject in need of such treatment.

For the purpose of this invention, the term “agonist” should be understood as including full agonists as well as partial agonists, whereby a “partial agonist” should be understood as a compound capable of partially, but not fully, activating GABA_{B} receptors.

The wording “TLESR”, transient lower esophageal sphincter relaxations, is herein defined in accordance with Mittal, R. K., Holloway, R. H., Penagini, R., Blackshaw, L. A., Dent, J., 1995; Transient lower esophageal sphincter relaxation. Gastroenterology 109, pp. 601-610.

The wording “reflux” is defined as fluid from the stomach being able to pass into the esophagus, since the mechanical barrier is temporarily lost at such times.

The wording “GERD”, gastroesophageal reflux disease, is defined in accordance with van Heerwarden, M. A., Smout A. J. P. M., 2000; Diagnosis of reflux disease. Baillière's Clin. Gastroenterol. 14, pp. 759-774.

A “combination” according to the invention may be present as a “fix combination” or as a “kit of parts combination”.

A “fix combination” is defined as a combination wherein (i) a compound of formula (I); and (ii) a GABA_{B} receptor agonist are present in one unit. One example of a “fix combination” is a pharmaceutical composition wherein (i) a compound of formula (I) and (ii) a GABA_{B} receptor agonist are present in admixture. Another example of a “fix combination” is a pharmaceutical composition wherein (i) a compound of formula (I) and (ii) a GABA_{B} receptor agonist; are present in one unit without being in admixture.

A “kit of parts combination” is defined as a combination wherein (i) a compound of formula (I) and (ii) a GABA_{B} receptor agonist are present in more than one unit. One example of a “kit of parts combination” is a combination wherein (i) a compound of formula (I) and (ii) a GABA_{B} receptor agonist are present separately. The components of the “kit of parts combination” may be administered simultaneously, sequentially or separately, i.e. separately or together.

The term “positive allosteric modulator” is defined as a compound which makes a receptor more sensitive to receptor agonists by binding to the receptor protein at a site different from that used by the endogenous ligand.

The term “therapy” and the term “treatment” also include “prophylaxis” and/or prevention unless stated otherwise. The terms “therapeutic” and “therapeutically” should be construed accordingly.

### Pharmaceutical Formulations

The compound of formula (I) can be formulated alone or in combination with a GABA_{B} receptor agonist.

For clinical use, the compound of formula (I), optionally in combination with a GABA_{B} receptor agonist, is in accordance with the present invention suitably formulated into pharmaceutical formulations for oral administration. Also rectal, parenteral or any other route of administration may be contemplated to the skilled man in the art of formulations. Thus, the compound of formula (I), optionally in combination with a GABA_{B} receptor agonist, is formulated with a pharmaceutically and pharmacologically acceptable carrier or adjuvant. The carrier may be in the form of a solid, semi-solid or liquid diluent.

In the preparation of oral pharmaceutical formulations in accordance with the invention, the compound of formula (I), optionally in combination with a GABA_{B} receptor agonist, to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of a compound of formula (I), optionally in combination with a GABA_{B} receptor agonist, with vegetable oil, fat, or other suitable vehicle for soft gelatine capsules. Hard gelatine capsules may contain a compound of formula (I), optionally in combination with a GABA_{B} receptor agonist, in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains a compound of formula (I), optionally in combination with a GABA_{B} receptor agonist, in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing a compound of formula (I), optionally in combination with a GABA_{B} receptor agonist, and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agents. Liquid preparations for oral administration may also be prepared in the form of a dry powder to be reconstituted with a suitable solvent prior to use.

Solutions for parenteral administration may be prepared as a solution of a compound of formula (I), optionally in combination with a GABA_{B} receptor agonist, in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, a compound of formula (I), optionally in combination with a GABA_{B} receptor agonist, may be administered once or twice daily, depending on the severity of the patient's condition. A typical daily dose of the compounds of formula (I) is from 0.1 to 100 mg per kg body weight of the subject to be treated, but this will depend on various factors such as the route of administration, the age and weight of the patient as well as of the severity of the patient's condition.

### Methods of Preparation

Hereinbelow, Scheme 1 denote methods for preparation of the compounds according to the present invention

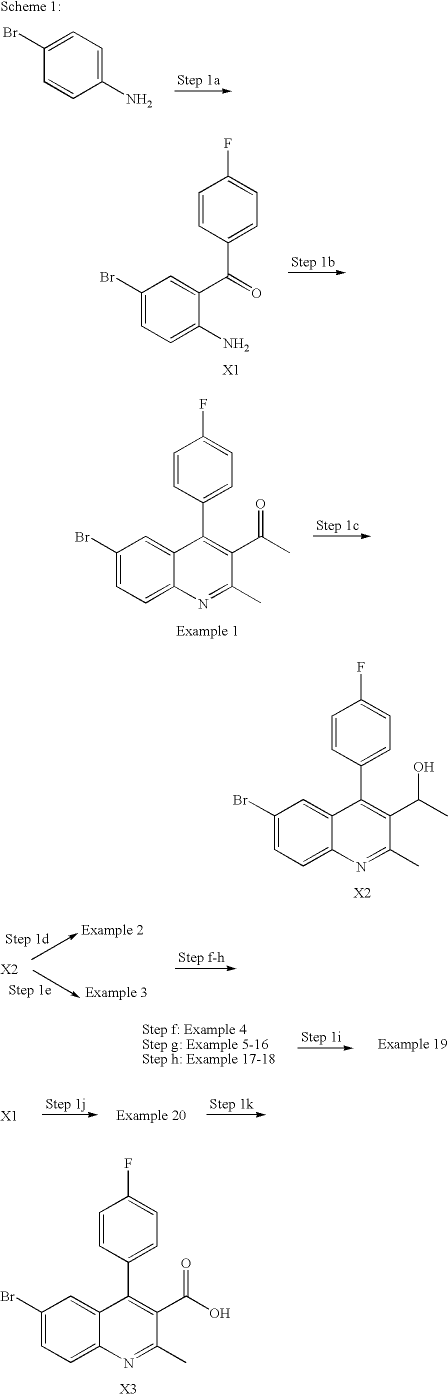

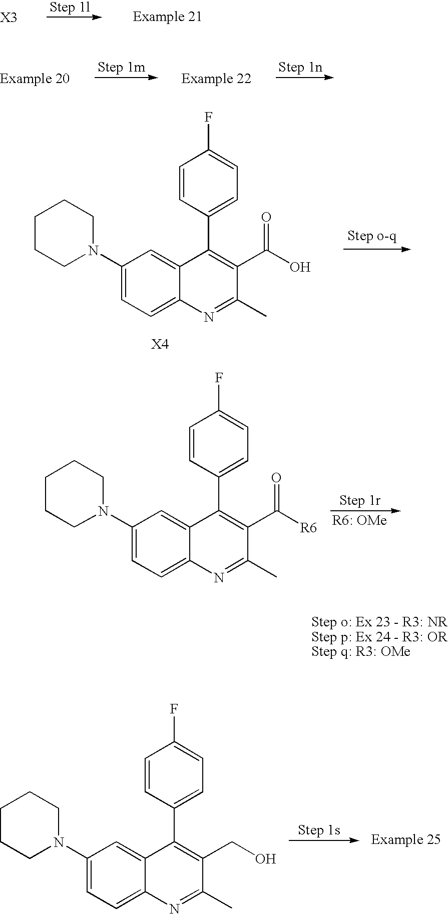

### EXAMPLES

### Abbreviations

DBU 2,3,4,6,7,8,9,10-octahydropyrimido[1,2-a]azepine
DCM dichloromethane

### DIPEA N,N-diisopropylethylamine

DMF N,N′-dimethylformamide
DMSO dimethylsulfoxide
EDC 1-ethyl-3-(3-dimethylaminopropyl)carbodiimide hydrochloride
EtOAc ethyl acetate
EtOH ethanol
FA formic acid
HOAt 1-hydroxy-7-azabenzotriazole
HPFC high performance flash chromatography
HPLC high performance liquid chromatography
LC-MS liquid chromatography mass spectroscopy
MeCN acetonitrile
MeOH methanol
NMR nuclear magnetic resonance
PyBOP benzotriazol-1-yl-oxytripyrrolidinophosphonium hexafluorophosphate
TBTU O-(benzotriazol-1-yl)-N,N,N′,N′-tetramethyluronium tetrafluoroborate
TEA triethylamine
TFA trifluoroacetic acid
THF tetrahydrofuran
UV ultra violet
atm atmosphere
rt room temperature
h hour(s)
min minutes
br broad
s singlet
d doublet
t triplet
q quartet
m multiplet
sep septet
dd double doublet
dt double triplet
td triple doublet

### General Experimental Procedures

Phase Separator from IST was used. Flash column chromatography employed normal phase silica gel 60 (0.040-0.063 mm, Merck) or IST Isolute®SPE columns normal phase silica gel or Fluorous SPE cartridges (FluoroFlash®SPE-cartridges) from Fluorous Technologies inc. or Biotage Horizon™ HPFC System using silica FLASH+™ HPFC™ Cartridges. HPLC purifications were performed on either a Gilson preparative HPLC system with gradient pump system 333/334, GX-281 injector, UV/VIS detector 155. Trilution LC v.1.4 software. In acidic system equipped with an Kromasil C8 10 μm 250×20 ID mm column or Kromasil C8 10 μm 250×50 ID mm column and as gradient: mobile phase (buffer): H₂O/MeCN/FA 95/5/0.2 and mobile phase (organic): MeCN. In neutral system equipped with an Kromasil C8 10 μm 250×20 ID mm column or Kromasil C8 10 μm 250×50 ID mm column and as gradient: mobile phase (buffer): MeCN/0,1M NH₄OAc 5/95 and mobile phase (organic): MeCN. In basic system system equipped with an XBridge C18 10 μm 250×19 ID mm column or XBridge C18 10 μm 250×50 ID mm column and as gradient: mobile phase (buffer): H₂O/MeCN/NH₃ 95/5/0.2 and mobile phase (organic): MeCN. Or on a Waters preparative HPLC system equipped with a Kromasil C8 10 mm 250 mm×21.2 mm column and a gradient mobile phase (buffer): MeCN/0,1M NH₄OAc 5/95 and mobile phase (organic): MeCN or on a Waters FractionLynx HPLC system with a mass triggered fraction collector, equipped with a Xbridge Prep C18 5μ 19 mm×150 mm column using MeCN/NH₃ buffer system with a gradient from 95% mobile phase A (0,2% NH₃ in water, pH10) to 95% mobile phase B (100% MeCN) unless otherwise stated. ¹H NMR and ¹³C NMR measurements were performed on a BRUKER ACP 300 or on a Varian Inova 400, 500 or 600 spectrometer, operating at ¹H frequencies of 300, 400, 500, 600 MHz, respectively, and ¹³C frequencies of 75, 100, 125 and 150 MHz, respectively. Chemical shifts are given in δ values (ppm) with the solvents used as internal standard, unless otherwise stated. Microwave heating was performed using single node heating in a Smith Creator or Emrys Optimizer from Personal Chemistry, Uppsala, Sweden. Mass spectral data were obtained using a Micromass LCT or Waters Q-T of micro system and, where appropriate, either positive ion data or negative ion data were collected.

Compound names generated by ACD/Name Release 9.0. Product Version: 9.04 (Build 6210, 20 Jul. 2005)

### Explanation to Plate-NMR:

*The solutions are taken from a concentrated sample dissolved in (CH₃)₂SO and are diluted with (CD₃)₂SO. Since a substantial amount of (CH₃)₂SO is present in the sample, first a pre-scan is run and analysed to automatically suppress the (CH₃)₂SO (2.54 ppm) and H₂O (3.3 ppm) peaks. This means that in this so-called wet1D experiment the intensity of peaks that reside in these areas around 3.3 ppm and 2.54 ppm are reduced. Furthermore impurities are seen in the spectrum which give rise to a triplet at 1.12 ppm, a singlet at 2.96 ppm and two multiplets between 2.76-2.70 ppm and 2.61-2.55 ppm. Most probably these impurities are dimethylsulfone and diethylsulfoxide.

Hereinbelow, it should be noted that Examples 1, 3, 21, 23, and 24 are for reference purposes only.

### Example 1

### Synthesis of 1-[6-bromo-4-(4-fluorophenyl)-2-methylquinolin-3-yl]ethanone was Synthesized According to Scheme 1

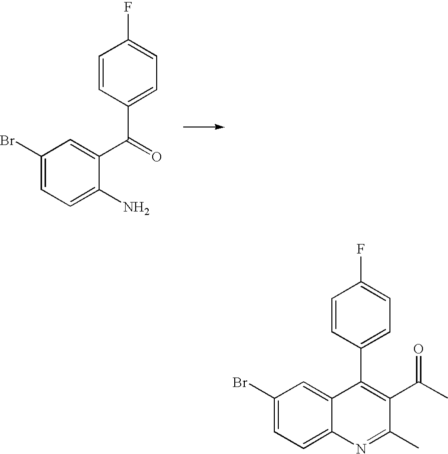

### Step 1a: Synthesis of (2-amino-5-bromophenyl)(4-fluorophenyl)methanone

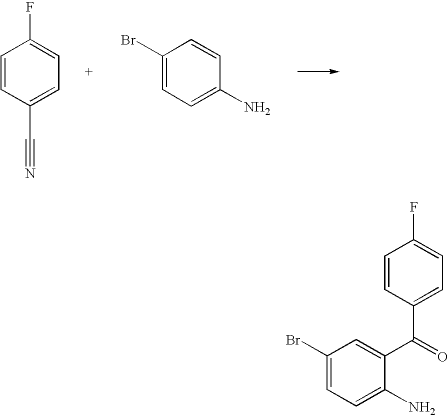

To a stirred solution of 79.24 g (0.594 mol) AlCl₃ in 300 ml anhydrous 1,2-dichloroethane were slowly added 119 g (0.594 mol) 4-bromoaniline in 100 ml 1,2-dichloroethane at 0° C. Then 63.81 g (0.545 mol) BCl₃ were added dropwise to the reaction mixture at −10° C. followed by addition of 60 g (0.495 mol) 4-bromo benzonitrile. The reaction mixture was warmed up to rt and refluxed for 18 h. The mixture was cooled to 0° C. and hydrolyzed by slowly adding water and subsequent heating at 80° C. for 1 h. The aq. layer was extracted with DCM and the combined organic layers were washed with water, brine, dried with Na₂SO₄ and concentrated in vacuo. The crude product was purified by flash column chromatography using 5% ethyl acetate in petrol ether as eluent to afford 13 g (44.2 mmol, 9%) of (2-amino-5-bromophenyl)(4-fluorophenyl)methanone as a yellow solid.

¹H NMR (400 MHz, CDCl₃) δ 5.98 (br s, 2H), 6.63 (d, 8.8 Hz, 2H), 7.14 (t, 8.7 Hz, 1H), 7.34 (dd, 8.8 Hz, 2.3 Hz, 1H), 7.49 (d, 2.3 Hz, 1H), 7.62-7.68 (m, 2H).

MS m/z 295.0 (M+H)⁺.

### Step 1b

### Example 1

### Synthesis of 1-[6-bromo-4-(4-fluorophenyl)-2-methylquinolin-3-yl]ethanone

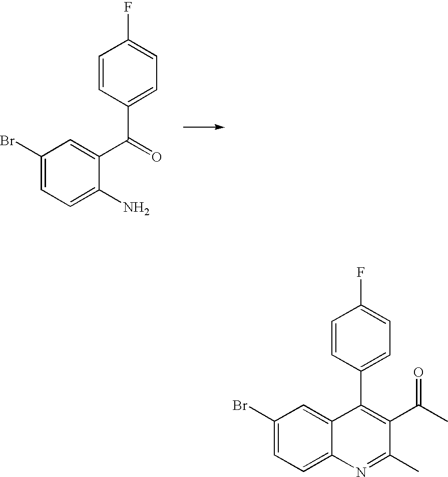

5 g (0.017 mol) of (2-amino-5-bromophenyl)(4-fluorophenyl)methanone, ref. step 1a product in Example 1, in 10 ml 2-propanol, were added to a solution of 3 g (0.0306 mol) 2,4 pentanedione and 100 mg (0.26 mmol) sodium tetrachloroaurate (III) dihydrate in 50 ml 2-propanol at rt. The mixture was heated to reflux for 18 h. The solvent was evaporated and the crude product purified by flash column chromatography with 4% ethyl acetate in petrol ether as eluent to afford 4.2 g (0.012 mol, 70%) of 1-[6-bromo-4-(4-fluorophenyl)-2-methylquinolin-3-yl]ethanone as a pale yellow solid.

¹H NMR (400 MHz, CDCl₃) δ 2.01 (s, 3H), 2.64 (s, 3H), 7.19-7.25 (m, 2H), 7.28-7.33 (m, 2H), 7.67 (d, 2.1 Hz, 1H), 7.77 (dd, 8.9 Hz, 2.2 Hz, 1H), 7.92 (d, 9 Hz, 1H).

MS m/z 359.0 (M+H)⁺.

### Example 2

### Synthesis of 6-bromo-3-[1-(4-chlorophenoxy)ethyl]-4-(4-fluorophenyl)-2-methylquinoline was Synthesized According to Scheme 1

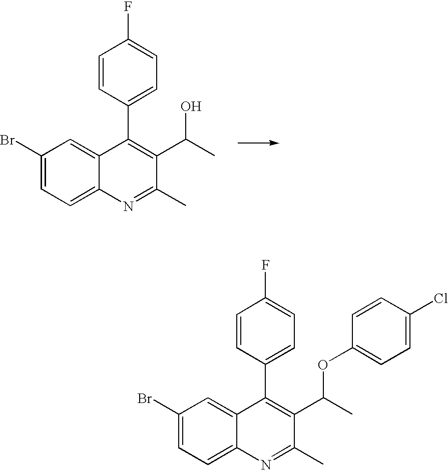

### Step 1c: Synthesis of 1-[6-bromo-4-(4-fluorophenyl)-2-methylquinolin-3-yl]ethanol

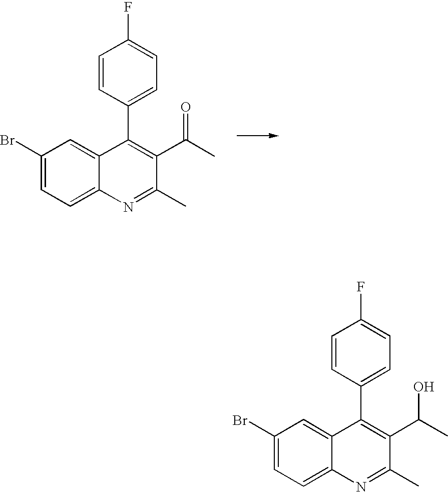

183 mg (0.51 mmol) of 1-[6-bromo-4-(4-fluorophenyl)-2-methylquinolin-3-yl]ethanone, ref Example 1, were suspended in 20 ml methanol and 39 mg (1.02 mmol) sodium borohydride was added portionwise. The solution became clear after stirring at rt for 3 h. The solvent was evaporated and the residue was partioned between DCM and water. The aq. phase was extracted with DCM and the combined organic layers were dried by filtration through a phase separator. The solvent was evaporated and the crude product could be used without further purification for the next step. 180.0 mg (0.50 mmol, 98%) 1-[6-bromo-4-(4-fluorophenyl)-2-methylquinolin-3-yl]ethanol was isolated as a white solid.

¹H NMR (400 MHz, CDCl₃) δ 1.50 (d, 7.0 Hz, 3H), 2.99 (s, 3H), 4.97-5.05 (m, 1H), 7.11-7.17 (m, 1H), 7.19-7.26 (m, 3H), 7.27-7.30 (m, 1H), 7.68 (dd, 8.9 Hz, 2.0 Hz, 1H), 7.85-7.95 (m, 1H).

MS m/z 361.0 (M+H)⁺.

### Step 1d

### Example 2

### Synthesis of 6-bromo-3-[1-(4-chlorophenoxy)ethyl]-4-(4-fluorophenyl)-2-methylquinoline

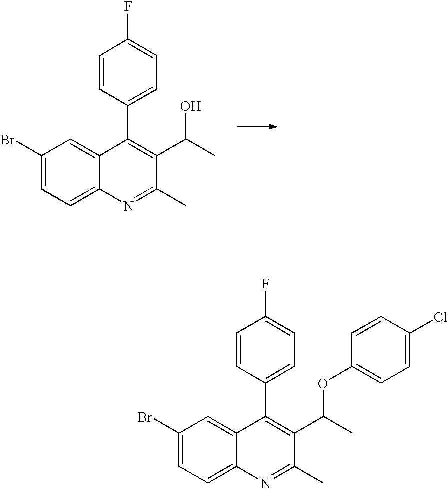

65 mg (0.18 mmol) of 1-[6-bromo-4-(4-fluorophenyl)-2-methylquinolin-3-yl]ethanol, ref step 1c product in Example 2, were dissolved in 1 ml anhydrous THF, then 52 mg (0.20 mmol) of triphenyl phosphine and 35 mg (0.27 mmol) of 4-chlorophenol were added and the solution was cooled to 0° C. 0.04 ml (0.208 mmol) diisopropyl azodicarboxylate were added dropwise and the mixture was allowed to warm up to rt. After stirring at rt for 4 h, the solvent was evaporated and the residue was purified by HPFC with pentane:MTBE=8:1 as eluent. 62 mg (0.13 mmol, 73%) 1-[6-bromo-4-(4-fluorophenyl)-2-methylquinolin-3-yl]ethanol was isolated as colorless oil.

¹H NMR (400 MHz, CDCl₃) δ 1.66-1.72 (m, 3H), 2.96 (s, 3H), 5.22-5.30 (m, 1H), 6.50-6.57 (m, 2H), 7.03-7.09 (m, 2H), 7.09-7.19 (m, 2H), 7.19-7.30 (m, 3H), 7.65-7.72 (m, 1H), 7.83-7.89 (m, 1H).

HRMS Calcd for [C₂₄H₁₈BrClFNO+H]⁺: 470.0323. Found: 470.0318.

### Step 1e

### Example 3

### Synthesis of 1-[4-(4-fluorophenyl)-2-methyl-6-piperidin-1-ylquinolin-3-yl]ethanol was synthesized according to Scheme 1

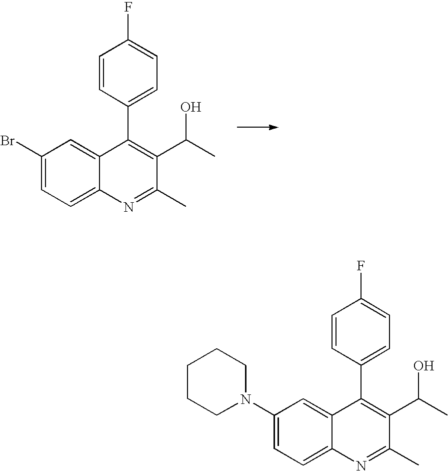

50 mg (0.054 mmol) of tris(dibenzylideneacetone)dipalladium(0) and 68 mg (0.11 mmol) of 2,2-bis(diphenylphosphino)-1,1-binaphtyl were dissolved in 4 ml anhydrous dioxane and 0.4 ml anhydrous tert-butanol under nitrogen atmosphere and were stirred at rt for 15 min. 390 mg (1.08 mmol) of 1-[6-bromo-4-(4-fluorophenyl)-2-methylquinolin-3-yl]ethanol, ref. step 1c product in Example 2, 182 mg (1.62 mmol) potassium tert-butoxide and 0.16 ml (1.62 mmol) piperidine were placed in a microwave reaction vessel under nitrogen atmosphere. The palladium catalyst solution was added and the vessel sealed and heated in the microwave oven at 130° C. for 1 h. The vessel was opened, the content was filtered and the filtrate was evaporated. The residue was purified by HPFC with pentane:ethyl acetate=1:2 gradient as eluent. 177 mg (0.49 mmol, 45%) 1-[4-(4-fluorophenyl)-2-methyl-6-piperidin-1-ylquinolin-3-yl]ethanol was isolated as a yellow solid.

¹H NMR (400 MHz, CDCl₃) δ 1.49 (d, 6.8 Hz, 3H), 1.50-1.55 (m, 2H), 1.59-1.66 (m, 4H), 2.93 (s, 3H), 2.98-3.03 (m, 4H), 4.98 (q, 6.8 Hz, 1H), 6.31 (d, 2.7 Hz, 1H), 7.12-7.25 (m, 4H), 7.40 (dd, 9.1 Hz, 2.6 Hz, 1H), 7.82-7.92 (m, 1H).

MS m/z 365.0 (M+H)⁺.

### Step 1f

### Example 4

### Synthesis of 3-[1-(4-chlorophenoxy)ethyl]-4-(4-fluorophenyl)-2-methyl-6-piperidin-1-ylquinoline was Synthesized According to Scheme 1

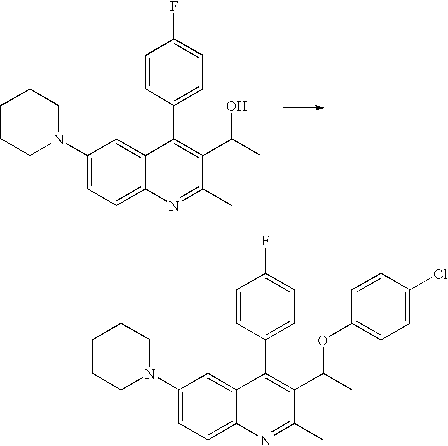

75 mg (0.21 mmol) of 1-[4-(4-fluorophenyl)-2-methyl-6-piperidin-1-ylquinolin-3-yl]ethanol, ref. Example 3, were dissolved in 0.5 ml anhydrous THF and 59 mg (0.23 mmol) triphenyl phosphine and 37 mg (0.29 mmol) 4-chlorophenol were added. The mixture was cooled to 0° C. and 0.048 ml (0.25 mmol) diisopropyl azodicarboxylate were added dropwise. The mixture was warmed up to rt and stirred at rt for 2 h. The solvent was evaporated and the crude product purified by HPFC with a gradient of pentane:ethyl acetate=5:1 to 3:1 as eluent. 68 mg (0.143 mmol, 70%) 3-[1-(4-chlorophenoxy)ethyl]-4-(4-fluorophenyl)-2-methyl-6-piperidin-1-ylquinoline was isolated as a yellow oil.

¹H NMR (400 MHz, CDCl₃) δ 1.47-1.55 (m, 2H), 1.58-1.65 (m, 4H), 1.68 (d, 6.8 Hz, 3H), 2.91 (s, 3H), 2.98-3.03 (m, 4H), 5.24 (q, 6.7 Hz, 1H), 6.29-6.32 (d, 2.5 Hz, 1H), 6.55 (d, 8.9 Hz, 2H), 7.05 (d, 8.9 Hz, 2H), 7.08-7.14 (m, 1H), 7.15-7.27 (m, 3H), 7.41 (dd, 9.2 Hz, 2.6 Hz, 1H), 7.86 (d, 9.2 Hz, 1H).

HRMS Calcd for [C₂₉H₂₈ClFN₂O+H]⁺: 475.1952. Found: 475.1943.

### Step 1g

### Example 5

### Synthesis of 4-(4-fluorophenyl)-2-methyl-6-piperidin-1-yl-3-[1-(pyridin-2-yloxy)ethyl]quinoline was Synthesized According to Scheme 1

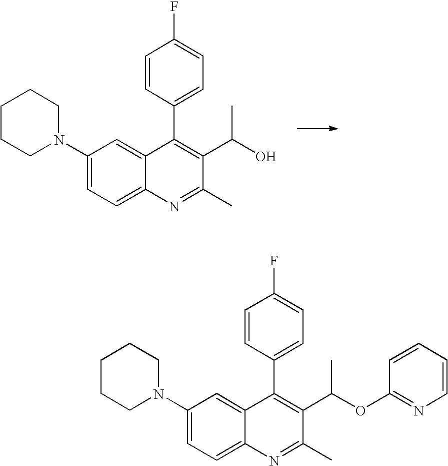

55 mg (0.15 mmol) of 1-[4-(4-fluorophenyl)-2-methyl-6-piperidin-1-ylquinolin-3-yl]ethanol, ref. Example 3, 163 mg (0.23 mmol) diphenyl-[4-(1H,1H,2H,2H-perfluorodecyl)phenyl]phosphine and 22 mg (0.23 mmol) 2-hydroxypyridine were dissolved in 1 ml anhydrous THF and cooled to 0° C. 100 mg (0.23 mmol) of a solution of diethylazodicarboxylate (40% in toluene) were added and the mixture was warmed up to rt and stirred at rt for 14 h. The solvent was evaporated and the residue dissolved in 0.4 ml DMF. This solution was applied on a Fluorous SPE cartridge (2 g, preconditioned with 1 ml DMF and 4 ml MeOH:H₂O=8:2). The product was eluted with 10 ml MeOH:H₂O=8:2, the solvents were evaporated and the residue purified by reverse phase preparative HPLC. 21 mg (0.026 mmol, 32%) of 4-(4-fluorophenyl)-2-methyl-6-piperidin-1-yl-3-[1-(pyridin-2-yloxy)ethyl]quinoline was isolated as a light yellow solid.

¹H NMR (600 MHz, DMSO-d₆) δ 1.42-1.57 (m, 2H), 1.49-1.54 (m, 4H), 1.57 (d, 6.9 Hz, 3H), 2.79 (s, 3H), 2.94-2.97 (m, 4H), 5.94 (q, 7.0 Hz, 1H), 6.20 (d, 2.5 Hz, 1H), 6.73 (d, 8.3 Hz, 1H), 6.84 (dd, 6.8 Hz, 5.4 Hz, 1H), 7.23-7.27 (m, 1H), 7.36 (dt, 8.7 Hz, 2.7 Hz, 1H), 7.42 (dt, 8.9 Hz, 2.7 Hz, 1H), 7.47 (dd, 9.2 Hz, 2.1 Hz, 1H), 7.50-7.53 (m, 1H), 7.60-7.63 (m, 1H), 7.69 (d, 9.3 Hz, 1H), 7.98 (dd, 5.0 Hz, 1.8 Hz, 1H).

HRMS Calcd for [C₂₈H₂₈FN₃O+H]⁺: 442.2295. Found: 442.2283.

The Following Examples 6-16 were Synthesized According to Example 5 (Employing the Appropriate Phenol-Derivative):

### Example 6

### Synthesis of 4-{1-[4-(4-fluorophenyl)-2-methyl-6-piperidin-1-ylquinolin-3-yl]ethoxy}benzonitrile

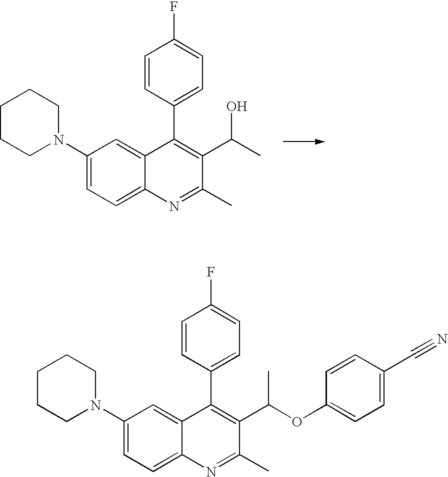

From 55 mg (0.15 mmol) of 1-[4-(4-fluorophenyl)-2-methyl-6-piperidin-1-ylquinolin-3-yl]ethanol, 12 mg (0.026 mmol, 17%) of the title compound was isolated.

¹H NMR (600 MHz, DMSO-d₆) δ 1.43-1.48 (m, 2H), 1.49-1.54 (m, 4H), 1.69 (d, 6.7 Hz, 3H), 2.76 (s, 3H), 2.97-3.00 (m, 4H), 5.31 (q, 6.7 Hz, 1H), 6.22 (d, 2.3 Hz, 1H), 6.75 (d, 9.0 Hz, 2H), 7.26-7.31 (m, 1H), 7.39 (dt, 8.7 Hz, 2.7 Hz, 1H), 7.45 (dt, 8.7 Hz, 2.7 Hz, 1H), 7.49-7.55 (m, 2H), 7.63 (d, 8.8 Hz, 2H), 7.72 (d, 9.3 Hz, 1H).

HRMS Calcd for [C₃₀H₂₈FN₃O+H]⁺: 466.2295. Found: 466.2303.

### Example 7

### Synthesis of 3-{1-[4-(4-fluorophenyl)-2-methyl-6-piperidin-1-ylquinolin-3-yl]ethoxy}benzonitrile

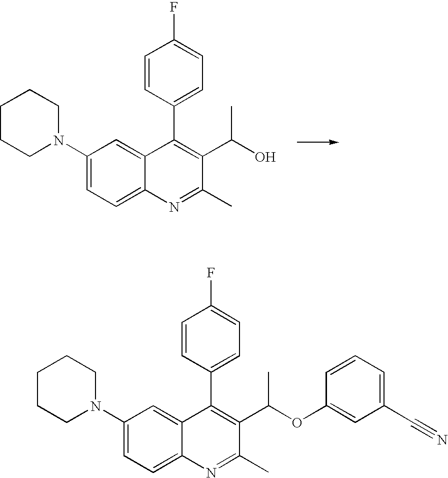

From 55 mg (0.15 mmol) of 1-[4-(4-fluorophenyl)-2-methyl-6-piperidin-1-ylquinolin-3-yl]ethanol, 27 mg (0.058 mmol, 38%) of the title compound was isolated.

¹H NMR (600 MHz, DMSO-d₆) δ 1.43-1.47 (m, 2H), 1.48-1.54 (m, 4H), 1.69 (d, 6.7 Hz, 3H), 2.79 (s, 3H), 2.96-3.00 (m, 4H), 5.28 (q, 6.7 Hz, 1H), 6.22 (d, 2.6 Hz, 1H), 6.95 (dd, 8.5 Hz, 2.2 Hz, 1H), 6.99-7.00 (m, 1H), 7.29 (d, 7.7 Hz, 1H), 7.31-7.38 (m, 3H), 7.39-7.45 (m, 2H), 7.51 (dd, 9.2 Hz, 2.5 Hz, 1H), 7.72 (d, 9.2 Hz, 1H).

HRMS Calcd for [C₃₀H₂₈FN₃O+H]⁺: 466.2295. Found: 466.2277.

### Example 8

### Synthesis of 4-(4-fluorophenyl)-2-methyl-3-{1-[4-(methylsulfonyl)phenoxy]ethyl}-6-piperidin-1-ylquinoline

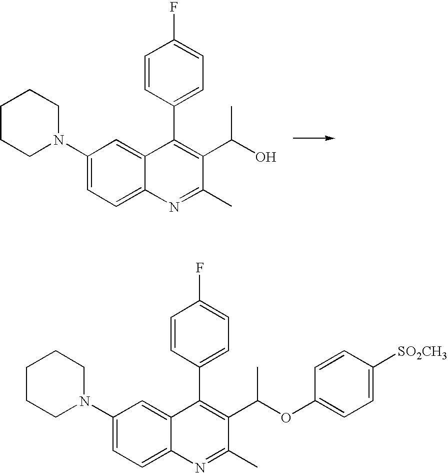

From 55 mg (0.15 mmol) of 1-[4-(4-fluorophenyl)-2-methyl-6-piperidin-1-ylquinolin-3-yl]ethanol, 5.3 mg (0.01 mmol, 7%) of the title compound was isolated.

¹H NMR (600 MHz, DMSO-d₆) δ 1.43-1.48 (m, 2H), 1.49-1.54 (m, 4H), 1.71 (d, 6.7 Hz, 3H), 2.76 (s, 3H), 2.97-3.00 (m, 4H), 3.04 (s, 3H), 5.30 (q, 6.6 Hz, 1H), 6.23 (d, 2.7 Hz, 1H), 6.81 (d, 8.9 Hz, 2H), 7.30-7.36 (m, 1H), 7.41 (dt, 8.6 Hz, 2.7 Hz, 1H), 7.47 (dt, 8.7 Hz, 2.7 Hz, 1H), 7.51 (dd, 9.2 Hz, 2.7 Hz, 1H), 7.57-7.61 (m, 1H), 7.70-7.74 (m, 3H).

HRMS Calcd for [C₃₀H₃₁FN₂O₃S+H]⁺: 519.2117. Found: 519.2114.

### Example 9

### Synthesis of 4-(4-fluorophenyl)-2-methyl-6-piperidin-1-yl-3-[1-(pyridin-3-yloxy)ethyl]quinoline

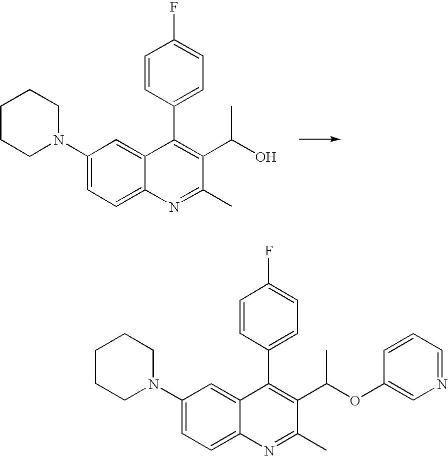

From 55 mg (0.15 mmol) of 1-[4-(4-fluorophenyl)-2-methyl-6-piperidin-1-ylquinolin-3-yl]ethanol, 29 mg (0.066 mmol, 44%) of the title compound was isolated.

¹H NMR (600 MHz, DMSO-d₆) δ 1.42-1.47 (m, 2H), 1.48-1.53 (m, 4H), 1.69 (d, 6.7 Hz, 3H), 2.79 (s, 3H), 2.96-2.99 (m, 4H), 5.26 (q, 6.8 Hz, 1H), 6.21 (d, 2.7 Hz, 1H), 6.94-6.97 (m, 1H), 7.27-7.30 (m, 1H), 7.26-7.36 (m, 1H), 7.34-7.40 (m, 2H), 7.44 (dt, 8.6 Hz, 2.7 Hz, 1H), 7.51 (dd, 9.2 Hz, 2.7 Hz, 1H), 7.72 (d, 9.2 Hz, 1H), 8.00 (d, 3.0 Hz, 1H), 8.05 (dd, 4.5 Hz, 1.2 Hz, 1H).

HRMS Calcd for [C₂₈H₂₈FN₃O+H]⁺: 442.2295. Found: 442.2286.

### Example 10

### Synthesis of 3-[1-(2-chlorophenoxy)ethyl]-4-(4-fluorophenyl)-2-methyl-6-piperidin-1-ylquinoline

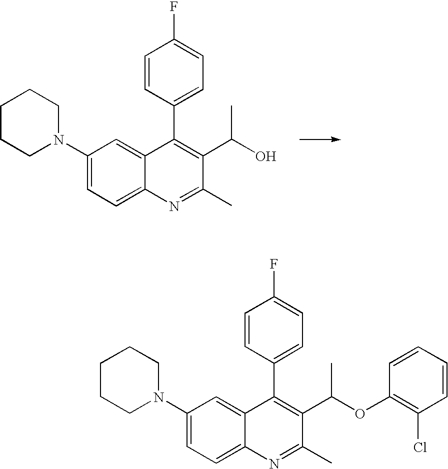

From 55 mg (0.15 mmol) of 1-[4-(4-fluorophenyl)-2-methyl-6-piperidin-1-ylquinolin-3-yl]ethanol, 23 mg (0.048 mmol, 32%) of the title compound was isolated.

¹H NMR (600 MHz, DMSO-d₆) δ 1.42-1.47 (m, 2H), 1.49-1.54 (m, 4H), 1.69 (d, 6.7 Hz, 3H), 2.85 (s, 3H), 2.96-2.99 (m, 4H), 5.22 (q, 6.7 Hz, 1H), 6.21 (d, 2.7 Hz, 1H), 6.50 (dd, 8.3 Hz, 1.3 Hz, 1H), 6.87 (dt, 7.6 Hz, 1.3 Hz, 1H), 7.08-7.11 (m, 1H), 7.23-7.27 (m, 1H), 7.29-7.33 (m, 1H), 7.30-7.40 (m, 3H), 7.51 (dd, 9.3 Hz, 2.7 Hz, 1H), 7.73 (d, 9.3 Hz, 1H).

HRMS Calcd for [C₂₉H₂₈ClFN₂O+H]⁺: 475.1952. Found: 475.1955.

### Example 11

### Synthesis of 4-(4-fluorophenyl)-3-[1-(4-methoxyphenoxy)ethyl]-2-methyl-6-piperidin-1-ylquinoline

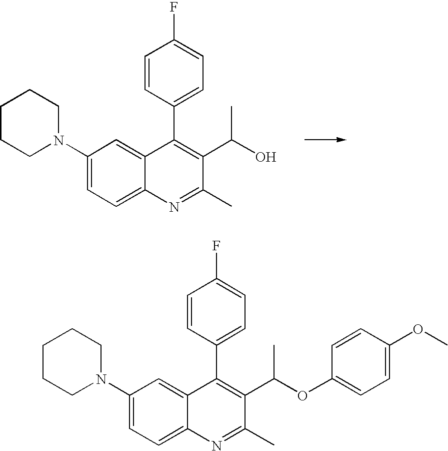

From 55 mg (0.15 mmol) of 1-[4-(4-fluorophenyl)-2-methyl-6-piperidin-1-ylquinolin-3-yl]ethanol, 30 mg (0.064 mmol, 43%) of the title compound was isolated.

¹H NMR (600 MHz, DMSO-d₆) δ 1.42-1.47 (m, 2H), 1.48-1.53 (m, 4H), 1.62 (d, 6.7 Hz, 3H), 2.80 (s, 3H), 2.95-2.98 (m, 4H), 3.58 (s, 3H), 5.07 (q, 6.9 Hz, 1H), 6.19 (d, 2.7 Hz, 1H), 6.54 (d, 9.1 Hz, 2H), 6.71 (d, 9.1 Hz, 2H), 7.25-7.28 (m, 1H), 7.29-7.32 (m, 1H), 7.37-7.44 (m, 2H), 7.49 (dd, 9.3 Hz, 2.4 Hz, 1H), 7.71 (d, 9.2 Hz, 1H).

HRMS Calcd for [C₃₀H₃₁FN₂O₂+H]⁺: 471.2448. Found: 471.2439.

### Example 12

### Synthesis of (3-{1-[4-(4-fluorophenyl)-2-methyl-6-piperidin-1-ylquinolin-3-yl]ethoxy}phenyl)dimethylamine

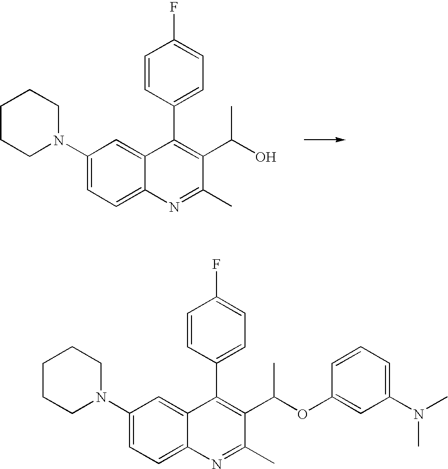

From 55 mg (0.15 mmol) of 1-[4-(4-fluorophenyl)-2-methyl-6-piperidin-1-ylquinolin-3-yl]ethanol, 35 mg (0.073 mmol, 49%) of the title compound was isolated.

¹H NMR (600 MHz, DMSO-d₆) δ 1.42-1.47 (m, 2H), 1.48-1.53 (m, 4H), 1.61 (d, 6.7 Hz, 3H), 2.75 (s, 6H), 2.81 (s, 3H), 2.95-2.98 (m, 4H), 5.17 (q, 6.7 Hz, 1H), 5.83 (dd, 8.1 Hz, 2.2 Hz, 1H), 5.96-5.98 (m, 1H), 6.19-6.22 (m, 2H), 6.89 (t, 8.2 Hz, 1H), 7.28-7.33 (m, 2H), 7.36-7.44 (m, 2H), 7.49 (dd, 9.3 Hz, 2.6 Hz, 1H), 7.71 (d, 9.3 Hz, 1H).

HRMS Calcd for [C₃₁H₃₄FN₃O+H]⁺: 484.2764. Found: 484.2768.

### Example 13

### Synthesis of 4-(4-fluorophenyl)-3-[1-(2-isopropylphenoxy)ethyl]-2-methyl-6-piperidin-1-ylquinoline

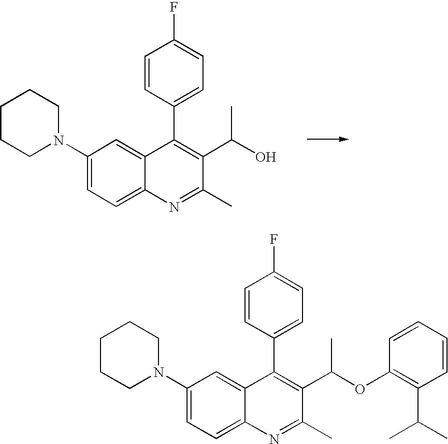

From 55 mg (0.15 mmol) of 1-[4-(4-fluorophenyl)-2-methyl-6-piperidin-1-ylquinolin-3-yl]ethanol, 31 mg (0.064 mmol, 43%) of the title compound was isolated.

¹H NMR (600 MHz, DMSO-d₆) δ 1.05 (d, 6.8 Hz, 3H), 1.08 (d, 6.9 Hz, 3H), 1.42-1.48 (m, 2H), 1.49-1.54 (m, 4H), 1.66 (d, 6.7 Hz, 3H), 2.82 (s, 3H), 2.96-2.99 (m, 4H), 3.12-3.19 (m, 1H), 5.11 (q, 6.7 Hz, 1H), 6.21 (d, 2.6 Hz, 1H), 6.34 (d, 8.2 Hz, 1H), 6.82 (t, 7.6 Hz, 1H), 6.89-6.93 (m, 1H), 7.13 (dd, 7.6 Hz, 1.6 Hz, 1H), 7.25-7.29 (m, 2H), 7.33-7.38 (m, 2H), 7.51 (dd, 9.2 Hz, 2.6 Hz, 1H), 7.74 (d, 9.3 Hz, 1H).

HRMS Calcd for [C₃₂H₃₅FN₂O+H]⁺: 483.2812. Found: 483.2832.

### Example 14

### Synthesis of 3-{1-[4-(4-fluorophenyl)-2-methyl-6-piperidin-1-ylquinolin-3-yl]ethoxy}benzamide

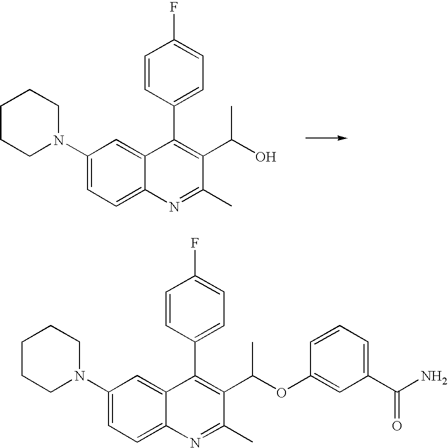

From 55 mg (0.15 mmol) of 1-[4-(4-fluorophenyl)-2-methyl-6-piperidin-1-ylquinolin-3-yl]ethanol, 39 mg (0.081 mmol, 54%) of the title compound was isolated.

¹H NMR (600 MHz, DMSO-d₆) δ 1.43-1.48 (m, 2H), 1.48-1.54 (m, 4H), 1.67 (d, 6.7 Hz, 3H), 2.77 (s, 3H), 2.96-2.99 (m, 4H), 5.21 (q, 6.9 Hz, 1H), 6.20 (d, 2.6 Hz, 1H), 6.81 (dd, 8.1 Hz, 2.6 Hz, 1H), 7.15-7.17 (m, 1H), 7.22 (t, 7.8 Hz, 1H), 7.26 (br s, 1H), 7.33 (d, 7.7 Hz, 2H), 7.38-7.42 (m, 2H), 7.49 (dd, 9.2 Hz, 2.8 Hz, 1H), 7.57-7.61 (m, 1H), 7.70 (d, 9.3 Hz, 1H), 7.84 (br s, 1H).

HRMS Calcd for [C₃₀H₃₀FN₃O₂+H]⁺: 484.2400. Found: 484.2400.

### Example 15

### Synthesis of 2-{1-[4-(4-fluorophenyl)-2-methyl-6-piperidin-1-ylquinolin-3-yl]ethoxy}benzonitrile

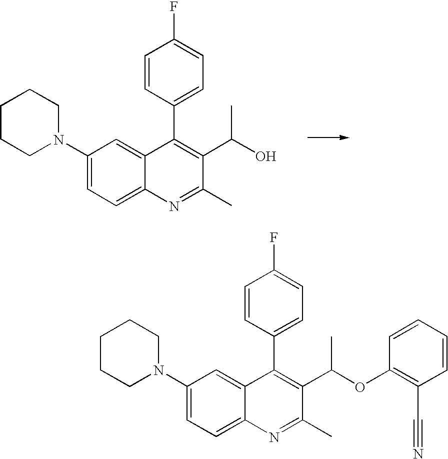

From 55 mg (0.15 mmol) of 1-[4-(4-fluorophenyl)-2-methyl-6-piperidin-1-ylquinolin-3-yl]ethanol, 24 mg (0.052 mmol, 34%) of the title compound was isolated.

¹H NMR (600 MHz, DMSO-d₆) δ 1.43-1.48 (m, 2H), 1.49-1.55 (m, 4H), 1.72 (d, 6.5 Hz, 3H), 2.82 (s, 3H), 2.97-3.00 (m, 4H), 5.37 (q, 6.8 Hz, 1H), 6.22 (d, 2.7 Hz, 1H), 6.56 (d, 8.7 Hz, 1H), 6.99 (t, 7.5 Hz, 1H), 7.26-7.30 (m, 1H), 7.38 (dt, 8.6 Hz, 2.7 Hz, 1H), 7.42 (dt, 8.6 Hz, 2.7 Hz, 1H), 7.45-7.48 (m, 2H), 7.52 (dd, 9.3 Hz, 2.6 Hz, 1H), 7.66 (dd, 7.6 Hz, 1.7 Hz, 1H), 7.73 (d, 9.2 Hz, 1H).

HRMS Calcd for [C₃₀H₂₈FN₃O+H]⁺: 466.2295. Found: 466.2290.

### Example 16

### Synthesis of 1-{1-[4-(4-fluorophenyl)-2-methyl-6-piperidin-1-ylquinolin-3-yl]ethyl}pyridin-2(1H)-one

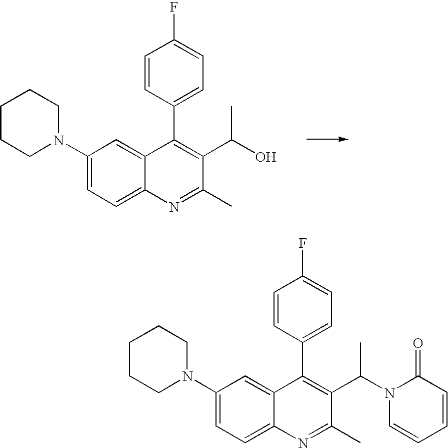

From 55 mg (0.15 mmol) of 1-[4-(4-fluorophenyl)-2-methyl-6-piperidin-1-ylquinolin-3-yl]ethanol, 4 mg (0.009 mmol, 6%) of the title compound was isolated.

¹H NMR (400 MHz, CDCl₃) δ 1.45-1.52 (m, 2H), 1.55-1.63 (m, 4H), 1.66 (d, 7.5 Hz, 3H), 2.74 (s, 3H), 2.92-2.97 (m, 4H), 5.82 (q, 7.41 Hz, 1H), 5.89 (dt, 6.9 Hz, 1H), 6.12 (d, 2.6 Hz, 1H), 6.39 (dd, 9.1 Hz, 0.8 Hz, 1H), 6.79-6.85 (m, 1H), 6.91-6.97 (m, 2H), 7.14-7.23 (m, 2H), 7.34-7.42 (m, 2H), 7.83 (d, 9.2 Hz, 1H).

HRMS Calcd for [C₂₈H₂₈FN₃O+H]⁺: 442.2295. Found: 442.2277.

### Example 17

### Synthesis of 3-[1-(4-chlorophenoxy)ethyl]-4-(4-fluorophenyl)-2-methyl-6-(4-methylpiperazin-1-yl)quinoline was synthesized according to Scheme 1

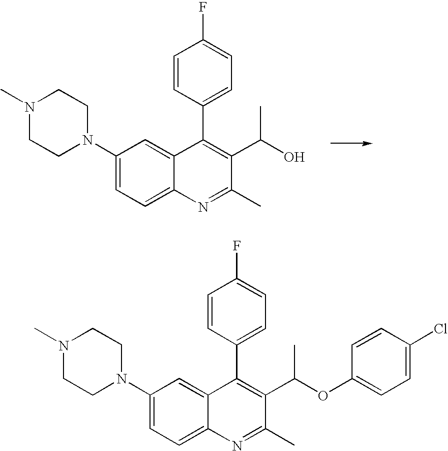

### Step 1e: Synthesis of 1-[4-(4-fluorophenyl)-2-methyl-6-(4-methylpiperazin-1-yl)quinolin-3-yl]ethanol was prepared as step 1e in Example 4

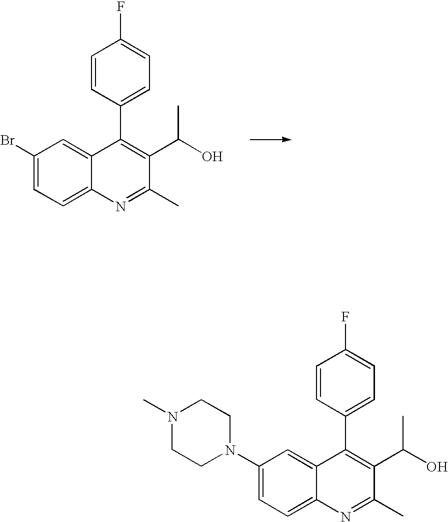

From 116 mg (0.322 mmol) of 1-[6-bromo-4-(4-fluorophenyl)-2-methylquinolin-3-yl]ethanol, ref step 1c product in Example 2, 66 mg (0.174 mmol, 54%) of 1-[4-(4-fluorophenyl)-2-methyl-6-(4-methylpiperazin-1-yl)quinolin-3-yl]ethanol was isolated as a yellow solid. The reaction mixture was heated at 120° C. for 30 min. The crude was purified by HPFC with a gradient of DCM:MeOH:NH₃ (26% in water)=20:1:0.05 to 10:1:0.05 as eluent.

¹H NMR (400 MHz, CDCl₃) δ 1.45 (d, 6.8 Hz, 3H), 2.25 (s, 3H), 2.43-2.48 (m, 4H), 2.89 (s, 3H), 2.97-3.02 (m, 4H), 4.93 (q, 6.8 Hz, 1H), 6.27 (d, 2.6 Hz, 1H), 7.01-7.19 (m, 4H), 7.32 (dd, 9.2 Hz, 2.6 Hz, 1H), 7.81 (d, 9.1 Hz, 1H).

MS m/z 380.0 (M+H)⁺.

### Step 1h

### Example 17

### Synthesis of 3-[1-(4-chlorophenoxy)ethyl]-4-(4-fluorophenyl)-2-methyl-6-(4-methylpiperazin-1-yl)quinoline

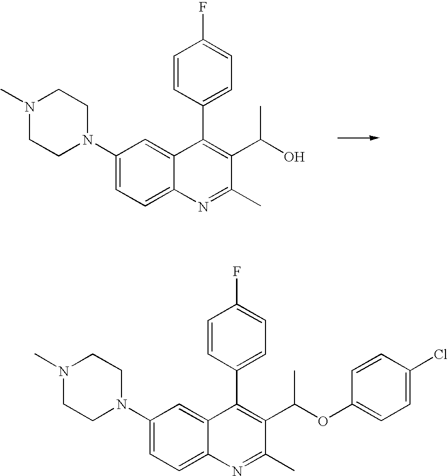

34 mg (0.089 mmol) of 1-[4-(4-fluorophenyl)-2-methyl-6-(4-methylpiperazin-1-yl)quinolin-3-yl]ethanol, ref. step 1e product in Example 17, 14 mg (0.107 mmol) of 4-chlorophenol and 28 mg (0.107 mmol) of triphenyl phosphine were dissolved in 1 ml anhydrous THF. The mixture was cooled to 0° C. and 48 mg (0.107 mmol) diethylazodicarboxylate (40% solution in toluene) were added dropwise. The mixture was warmed up to rt and stirred for 3 h. The solvent was removed in vacuo and the residue purified by reverse phase preparative HPLC. 32 mg (0.065 mmol, 74%) of 3-[1-(4-chlorophenoxy)ethyl]-4-(4-fluorophenyl)-2-methyl-6-(4-methylpiperazin-1-yl)quinoline was isolated as a light yellow solid.

¹H NMR (400 MHz, CDCl₃) δ 1.68 (d, 6.7 Hz, 3H), 2.29 (s, 3H), 2.47-2.51 (m, 4H), 2.91 (s, 3H), 3.04-3.08 (m, 4H), 5.24 (q, 6.8 Hz, 1H), 6.32 (d, 2.5 Hz, 1H), 6.55 (d, 8.9 Hz, 2H), 7.04 (d, 8.9 Hz, 2H), 7.08-7.25 (m, 2H), 7.26-7.33 (m, 2H), 7.39 (dd, 9.2 Hz, 2.6 Hz, 1H), 7.86 (d, 9.2 Hz, 1H).

HRMS Calcd for [C₂₉H₂₉ClFN₃O+H]⁺: 490.2061. Found: 490.2047.

Following Example 18 were Synthesized According to Example 17:

### Example 18

### Synthesis of 4-(4-fluorophenyl)-3-[1-(2-isopropylphenoxy)ethyl]-2-methyl-6-(4-methylpiperazin-1-yl)quinoline

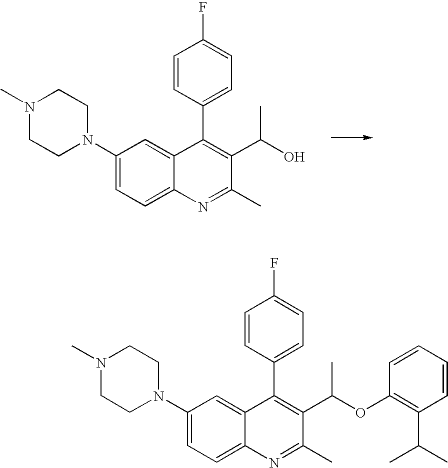

From 32 mg (0.084 mmol) of 1-[4-(4-fluorophenyl)-2-methyl-6-(4-methylpiperazin-1-yl)quinolin-3-yl]ethanol, ref. step 1e product in Example 17, 18 mg (0.036 mmol, 43%) of 4-(4-fluorophenyl)-3-[1-(2-isopropylphenoxy)ethyl]-2-methyl-6-(4-methylpiperazin-1-yl)quinoline was isolated as a yellow solid.

¹H NMR (400 MHz, CDCl₃) δ 1.10 (d, 7.0 Hz, 3H), 1.13 (d, 7.0 Hz, 3H), 1.68 (d, 6.7 Hz, 3H), 2.35 (s, 3H), 2.61-2.66 (m, 4H), 2.97 (s, 3H), 3.08-3.13 (m, 4H), 3.16-3.27 (m, 1H), 5.23 (q, 6.8 Hz, 1H), 6.34 (d, 2.6 Hz, 1H), 6.37 (dd, 7.8 Hz, 1.5 Hz, 1H), 6.80-6.89 (m, 2H), 7.05-7.20 (m, 5H), 7.38 (dd, 9.3 Hz, 2.7 Hz, 1H), 7.92 (d, 9.4 Hz, 1H).

HRMS Calcd for [C₃₂H₃₆FN₃O+H]⁺: 498.2921. Found: 498.2921.

### Example 19

### Synthesis of 4-{1-[4-(4-fluorophenyl)-2-methyl-6-piperidin-1-ylquinolin-3-yl]ethoxy}phenol was synthesized according to Scheme 1

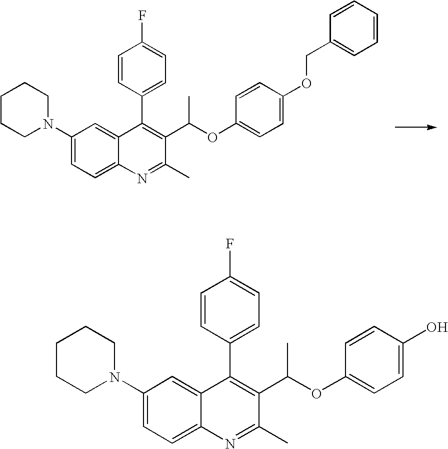

### Step 1g: Synthesis of 3-{1-[4-(benzyloxy)phenoxy]ethyl}-4-(4-fluorophenyl)-2-methyl-6-piperidin-1-ylquinoline was Prepared as in Ref. Example 5

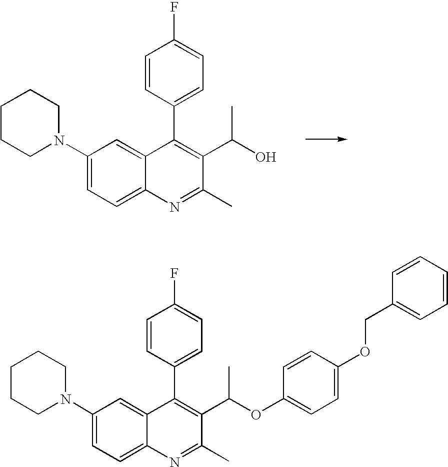

From 55 mg (0.15 mmol) of 1-[4-(4-fluorophenyl)-2-methyl-6-piperidin-1-ylquinolin-3-yl]ethanol, ref. Example 3, 39 mg (0.071 mmol, 47%) of the title compound was isolated.

¹H NMR (600 MHz, DMSO-d₆) δ 1.42-1.47 (m, 2H), 1.48-1.53 (m, 4H), 1.62 (d, 6.7 Hz, 3H), 2.81 (s, 3H), 2.95-2.98 (m, 4H), 4.91 (s, 2H), 5.07 (q, 6.7 Hz, 1H), 6.19 (d, 2.7 Hz, 1H), 6.54 (d, 9.1 Hz, 2H), 6.79 (d, 9.2 Hz, 2H), 7.21-7.24 (m, 1H), 7.24-7.28 (m, 1H), 7.28-7.35 (m, 5H), 7.36-7.40 (m, 2H), 7.49 (dd, 9.3 Hz, 2.7 Hz, 1H), 7.71 (d, 9.2 Hz, 1H).

HRMS Calcd for [C₃₆H₃₅FN₂O₂+H]⁺: 547.2761. Found: 547.2773.

### Step 1i

### Example 19

### Synthesis of 4-{1-[4-(4-fluorophenyl)-2-methyl-6-piperidin-1-ylquinolin-3-yl]ethoxy}phenol

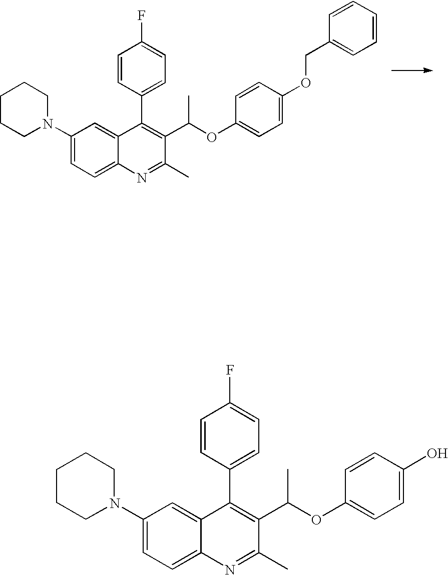

19 mg (0.035 mmol) of 3-{1-[4-(benzyloxy)phenoxy]ethyl}-4-(4-fluorophenyl)-2-methyl-6-piperidin-1-ylquinoline, ref step 1g product in Example 19, were dissolved in 3 ml methanol under a nitrogen atmosphere. 2 mg (0.014 mmol) Pd(OH)₂, 20 wt. % Pd (on dry basis) on carbon (wet) was added and the atmosphere was replaced by a hydrogen atmosphere. The mixture was stirred at atmospheric pressure and rt for 1 h. The catalyst was removed by filtration and the solvent was evaporated. The crude product was purified by HPFC with pentane:ethyl acetate=3:2 as eluent. 12 mg (0.026 mmol, 76%) of 4-{1-[4-(4-fluorophenyl)-2-methyl-6-piperidin-1-ylquinolin-3-yl]ethoxy}phenol was isolated as light yellow oil.

¹H NMR (400 MHz, CDCl₃) δ 1.45-1.53 (m, 2H), 1.56-1.62 (m, 4H), 1.63 (d, 6.7 Hz, 3H), 2.95 (s, 3H), 2.95-3.00 (m, 4H), 5.12 (q, 6.8 Hz, 1H), 6.26 (d, 2.8 Hz, 1H), 6.42 (d, 9.0 Hz, 2H), 6.47 (d, 9.0 Hz, 2H), 6.91-6.97 (m, 1H), 7.03-7.09 (m, 1H), 7.12-7.20 (m, 2H), 7.36 (dd, 9.2 Hz, 2.7 Hz, 1H), 7.85 (d, 9.3 Hz, 1H).

HRMS Calcd for [C₂₉H₂₉FN₂O₂+H]⁺: 457.2291. Found: 457.2282.

### Step 1j

### Example 20

### Synthesis of tert-butyl 6-bromo-4-(4-fluorophenyl)-2-methylquinoline-3-carboxylate was Synthesized According to Scheme 1

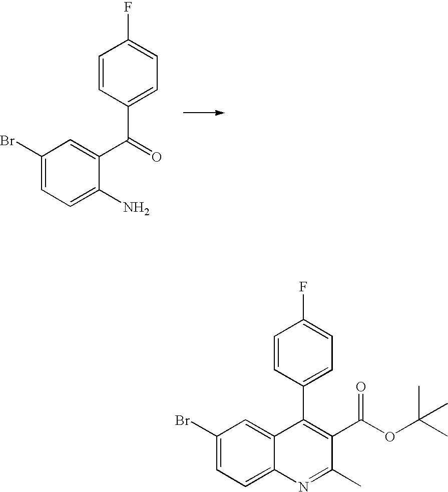

5 g (0.017 mol) of (2-amino-5-bromophenyl)(4-fluorophenyl)methanone, ref. step 1a product in example 1, in 10 ml 2-propanol, were added to a solution of 4 g (2.5 mmol) tert-butylacetylacetone and 169 mg (0.04 mmol) sodium tetrachloroaurate (III) dihydrate in 50 ml 2-propanol at rt and heated to reflux for 18 h. The reaction mixture was concentrated in vacuo and purified by flash column chromatography with 4% ethyl acetate in petrol ether as eluent to afford 1.4 g (3.4 mmol, 20%) tert-butyl 6-bromo-4-(4-fluorophenyl)-2-methylquinoline-3-carboxylate as a pale yellow solid.

¹H NMR (400 MHz, CDCl₃) δ 1.26 (s, 9H), 2.74 (s, 3H), 7.16-7.23 (m, 2H), 7.29-7.34 (m, 2H), 7.59 (d, 2.1 Hz, 1H), 7.75 (dd, 8.9 Hz, 2.2 Hz, 1H), 7.91 (d, 8.9 Hz, 1H).

MS m/z 416.0 (M+H)⁺.

### Example 21

### Synthesis of 6-bromo-4-(4-fluorophenyl)-2-methyl-3-(piperidin-1-ylcarbonyl)quinoline was Synthesized According to Scheme 1

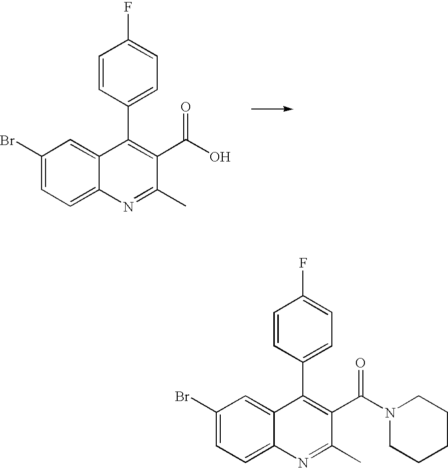

### Step 1k

### Synthesis of 6-bromo-4-(4-fluorophenyl)-2-methylquinoline-3-carboxylic acid

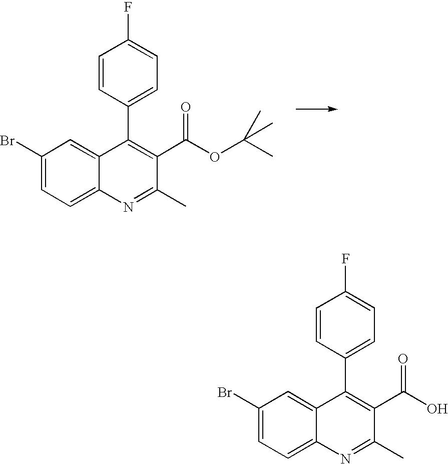

141 mg (0.34 mmol) of tert-butyl 6-bromo-4-(4-fluorophenyl)-2-methylquinoline-3-carboxylate, ref. example 20, were dissolved in 1 ml DCM and 1 ml TFA was added. The mixture was warmed up to 40° C. and stirred at that temperature for 3 h. The solvents were removed in vacuo and excessive TFA was removed by co-evaporation with toluene. The crude product could be used in the next steps without further purification. 118 mg (0.328 mmol, 97%) of 6-bromo-4-(4-fluorophenyl)-2-methylquinoline-3-carboxylic acid was isolated as a colorless oil.

¹H NMR (400 MHz, DMSO-d₆) δ 2.62 (s, 3H), 7.32-7.43 (m, 4H), 7.47 (d, 2.0 Hz, 1H), 7.86 (dd, 8.9 Hz, 2.1 Hz, 1H), 7.93 (d, 8.9 Hz, 1H).

HRMS Calcd for [C₁₇H₁₁BrFNO₂+H]⁺: 360.0035. Found: 360.0019.

### Step 1l

### Example 21

### Synthesis of 6-bromo-4-(4-fluorophenyl)-2-methyl-3-(piperidin-1-ylcarbonyl)quinoline

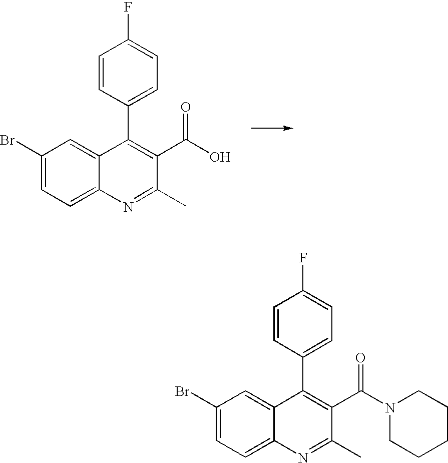

41 mg (0.114 mmol) of 6-bromo-4-(4-fluorophenyl)-2-methylquinoline-3-carboxylic acid, ref. step 1k product in Example 21, were dissolved in 0.6 ml anhydrous DMF and 73 mg (0.23 mmol) TBTU and 0.04 ml (0.228 mmol) DIPEA were added. After stirring at rt for 10 min, 0.022 ml (0.23 mmol) piperidine were added and the mixture was stirred at rt for 3 h. 0.1M aq. HCl solution was added and the aq. phase was extracted with DCM. The combined organic phases were washed with NaHCO₃ solution and dried by filtration through a phase separator. The solvent was evaporated and the residue purified by HPFC with a gradient of pentane:ethyl acetate=2:3 to 1:2 as eluent. 37 mg (0.087 mmol, 76%) of 6-bromo-4-(4-fluorophenyl)-2-methyl-3-(piperidin-1-ylcarbonyl)quinoline was isolated as a colorless oil.

¹H NMR (400 MHz, CDCl₃) δ 0.95-1.06 (m, 1H), 1.17-1.33 (m, 2H), 1.36-1.44 (m, 1H), 1.44-1.54 (m, 2H), 2.67 (s, 3H), 2.73-2.80 (m, 1H), 2.98-3.05 (m, 1H), 3.29-3.37 (m, 1H), 3.52-3.60 (m, 1H), 7.14-7.29 (m, 3H), 7.50-7.57 (m, 1H), 7.68 (d, 2.1 Hz, 1H), 7.73 (dd, 8.9 Hz, 2.1 Hz, 1H), 7.90 (d, 8.9 Hz, 1H).

HRMS Calcd for [C₂₂H₂₀BrFN₂O+H]⁺: 427.0821. Found: 427.0801.

### Step 1m

### Example 22

### Synthesis of tert-butyl 4-(4-fluorophenyl)-2-methyl-6-piperidin-1-ylquinoline-3-carboxylate was synthesized according to Scheme 1

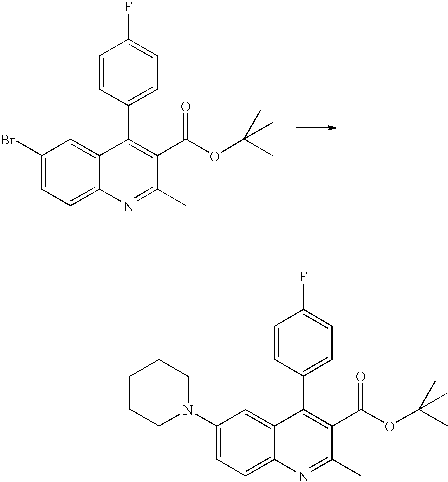

55.5 mg (0.061 mmol) tris(dibenzylideneacetone)dipalladium(0) and 76 mg (0.121 mmol) 2,2-bis(diphenylphosphino)-1,1-binaphtyl were dissolved in 4 ml anhydrous dioxane and 0.4 ml anhydrous tert-butanol under nitrogen atmosphere and the mixture was stirred at rt for 15 min. 505 mg (1.21 mmol) of tert-butyl 6-bromo-4-(4-fluorophenyl)-2-methylquinoline-3-carboxylate, ref. Example 20, were placed into a microwave reaction vessel and 204 mg (1.82 mmol) potassium tert-butoxide and 0.24 ml (2.43 mmol) piperidine were added under nitrogen atmosphere. The vessel was sealed and the catalyst solution added with a syringe. The vessel was heated in the microwave oven at 120° C. for 30 min. The vessel was opened, the content filtrated and the filtrate was evaporated. The residue was purified by HPFC with pentane:ethyl acetate=5:1 as eluent. 196 mg (0.47 mmol, 38%) tert-butyl 4-(4-fluorophenyl)-2-methyl-6-piperidin-1-ylquinoline-3-carboxylate was isolated as a yellow oil.

¹H NMR (400 MHz, CDCl₃) δ 1.25 (s, 9H), 1.50-1.56 (m, 2H), 1.61-1.68 (m, 4H), 2.70 (s, 3H), 3.04-3.10 (m, 4H), 6.63 (d, 2.6 Hz, 1H), 7.14-7.20 (m, 2H), 7.30-7.36 (m, 2H), 7.46 (dd, 9.3 Hz, 2.6 Hz, 1H), 7.89 (d, 8.9 Hz, 1H).

HRMS Calcd for [C₂₆H₂₉FN₂O₂+H]⁺: 421.2291. Found: 421.2285.

### Example 23

### Synthesis of 4-(4-fluorophenyl)-2-methyl-6-piperidin-1-yl-3-(piperidin-1-ylcarbonyl)quinoline was Synthesized According to Scheme 1

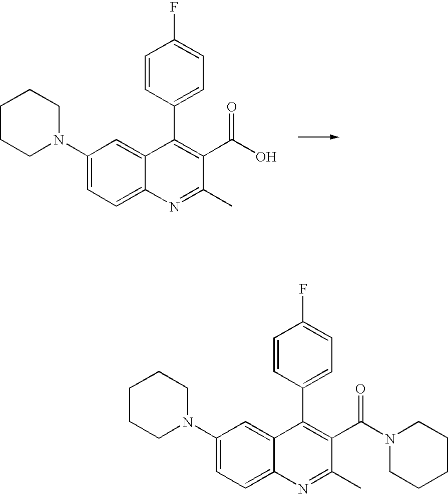

### Step 1n: Synthesis of 4-(4-fluorophenyl)-2-methyl-6-piperidin-1-ylquinoline-3-carboxylic acid

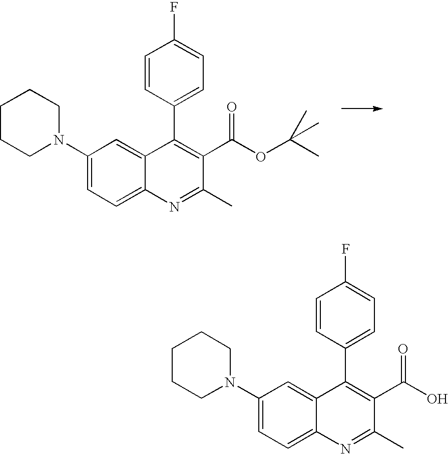

22 mg (0.052 mmol) of tert-butyl 4-(4-fluorophenyl)-2-methyl-6-piperidin-1-ylquinoline-3-carboxylate, ref. Example 22, were dissolved in 0.5 ml DCM and 0.5 ml TFA were added. The mixture was heated to 50° C. and stirred for 30 min. The solvents were evaporated and excessive TFA was removed by co-evaporation with toluene. The crude product was used without further purification for the next steps. 19 mg (0.05 mmol, 96%) 4-(4-fluorophenyl)-2-methyl-6-piperidin-1-ylquinoline-3-carboxylic acid was obtained as a light yellow oil.

¹H NMR (400 MHz, DMSO-d₆) δ 1.45-1.60 (m, 6H), 2.65 (s, 3H), 3.07-3.17 (m, 4H), 6.58-6.65 (m, 1H), 7.07-7.16 (m, 1H), 7.18-7.24 (m, 1H), 7.33-7.43 (m, 2H), 7.73 (d, 9.1 Hz, 1H), 7.89 (d, 9.2 Hz, 1H).

MS m/z 365.0 (M+H)⁺.

### Step 1o

### Example 23

### Synthesis of 4-(4-fluorophenyl)-2-methyl-6-piperidin-1-yl-3-(piperidin-1-ylcarbonyl)quinoline

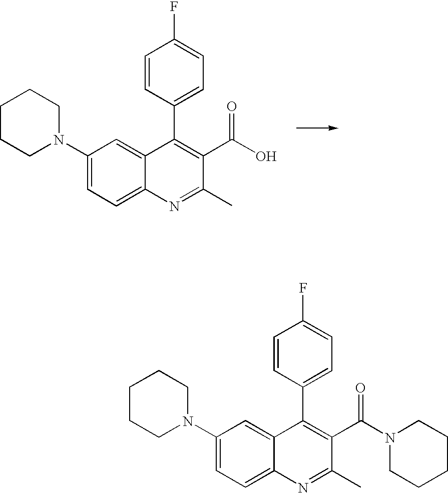

19 mg (0.05 mmol) 4-(4-fluorophenyl)-2-methyl-6-piperidin-1-ylquinoline-3-carboxylic acid, ref step 1n product in Example 23, was dissolved in 0.5 ml anhydrous DMF and 25 mg (0.078 mmol) TBTU and 0.014 ml (0.078 mmol) DIPEA were added. The mixture was stirred at rt for 10 min and 0.01 ml (0.104 ml) piperidine were added. After stirring at rt for 3 h, 0.1 M aq. HCl solution was added and the aq. phase was extracted with DCM. The combined organic layers were washed with NaHCO₃ solution and dried by filtration through a phase separator. After evaporation of the solvent, the residue was purified by HPFC with a gradient of pentane:ethyl acetate=2:3 to 1:2 as eluent. 15 mg (0.035 mmol, 67%) of 4-(4-fluorophenyl)-2-methyl-6-piperidin-1-yl-3-(piperidin-1-ylcarbonyl)-quinoline was isolated as a yellow solid.

¹H NMR (400 MHz, CDCl₃) δ 1.02-1.10 (m, 1H), 1.22-1.36 (m, 2H), 1.38-1.48 (m, 2H), 1.49-1.60 (m, 3H), 1.65-1.72 (m, 4H), 2.67 (s, 3H), 2.82-2.86 (m, 1H), 3.05-3.15 (m, 5H), 3.31-3.39 (m, 1H), 3.57-3.64 (m, 1H), 6.77 (d, 2.6 Hz, 1H), 7.15-7.25 (m, 2H), 7.32-7.36 (m, 1H), 7.50 (dd, 9.2 Hz, 2.6 Hz, 1H), 7.56-7.60 (m, 1H), 7.93 (d, 9.2 Hz, 1H).

HRMS Calcd for [C₂₇H₃₀FN₃O+H]⁺: 432.2451. Found: 432.2457.

### Step 1p

### Example 24

### Synthesis of 3,3,3-trifluoropropyl 4-(4-fluorophenyl)-2-methyl-6-piperidin-1-ylquinoline-3-carboxylate was synthesized according to Scheme 1

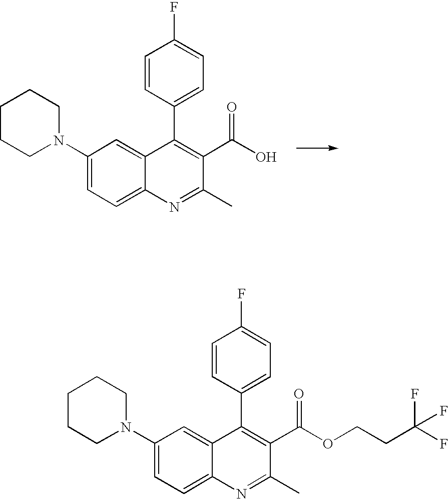

29 mg (0.08 mmol) of 4-(4-fluorophenyl)-2-methyl-6-piperidin-1-ylquinoline-3-carboxylic acid, ref. step in product in Example 23, were dissolved in 2 ml DCM. 21 mg (0.11 mmol) EDC, 18 mg (0.143 mmol) 4-dimethylaminopyridine and 11 mg (0.095 mmol) 3,3,3-trifluoropropano-1-ol were added and the mixture was stirred at rt for 3 days. 0.1 M aq. HCl solution was added and the aq. phase was extracted with DCM. The combined organic layers were washed with NaHCO₃ solution and dried by filtration through a phase separator. The solvent was evaporated and the crude product purified by HPFC with pentane:ethyl acetate=4:1 as eluent. 26 mg (0.056 mmol, 71%) 3,3,3-trifluoropropyl 4-(4-fluorophenyl)-2-methyl-6-piperidin-1-ylquinoline-3-carboxylate was isolated as a light yellow oil.

¹H NMR (400 MHz, CDCl₃) δ 1.50-1.57 (m, 2H), 1.61-1.68 (m, 4H), 2.10-2.22 (m, 2H), 2.68 (s, 3H), 3.05-3.11 (m, 4H), 4.18 (t, 6.4 Hz, 2H), 6.65 (d, 2.6 Hz, 1H), 7.14-7.20 (m, 2H), 7.29-7.34 (m, 2H), 7.50 (dd, 9.4 Hz, 2.7 Hz, 1H), 7.91 (d, 9.1 Hz, 1H).

HRMS Calcd for [C₂₅H₂₄F₄N₂O₂+H]⁺: 461.1852. Found: 461.1848.

### Example 25

### Synthesis of 3-[(4-chlorophenoxy)methyl]-4-(4-fluorophenyl)-2-methyl-6-piperidin-1-ylquinoline was synthesized according to Scheme 1

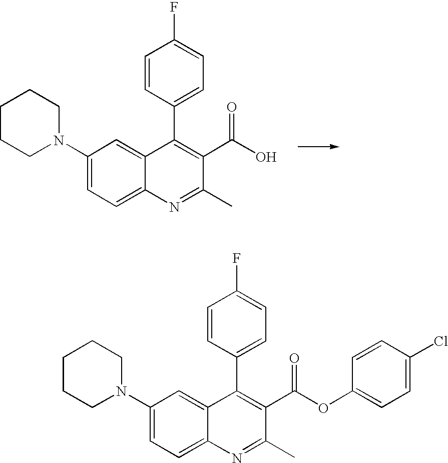

### Step 1q: Synthesis of methyl 4-(4-fluorophenyl)-2-methyl-6-piperidin-1-ylquinoline-3-carboxylate

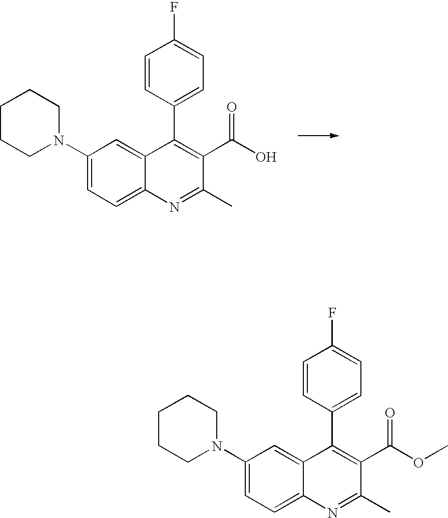

55 mg (0.151 mmol) of 4-(4-fluorophenyl)-2-methyl-6-piperidin-1-ylquinoline-3-carboxylic acid, ref. Example 23 step 1n product, were dissolved in 5 ml DCM and 0.113 ml (0.23 mmol) of a 2 M solution of trimethylsilyldiazomethane in DCM were added. The mixture was stirred at rt for 3 h. 0.09 ml (1.5 mmol) acetic acid were added and the mixture was stirred for another hour at rt. The solvents were removed in vacuo and the crude product purified by HPFC with pentane:ethyl acetate=3:1 as eluent. 51 mg (0.135 mmol, 89%) of methyl 4-(4-fluorophenyl)-2-methyl-6-piperidin-1-ylquinoline-3-carboxylate was isolated as a light yellow oil.

¹H NMR (400 MHz, CDCl₃) δ 1.49-1.56 (m, 2H), 1.60-1.67 (m, 4H), 2.67 (s, 3H), 3.05-3.09 (m, 4H), 3.56 (s, 3H), 6.67 (d, 2.6 Hz, 1H), 7.12-7.18 (m, 2H), 7.28-7.34 (m, 2H), 7.47 (dd, 9.3 Hz, 2.7 Hz, 1H), 7.89 (d, 9.2 Hz, 1H).

MS m/z 379.0 (M+H)⁺.

### Step 1r: Synthesis of [4-(4-fluorophenyl)-2-methyl-6-piperidin-1-ylquinolin-3-yl]methanol

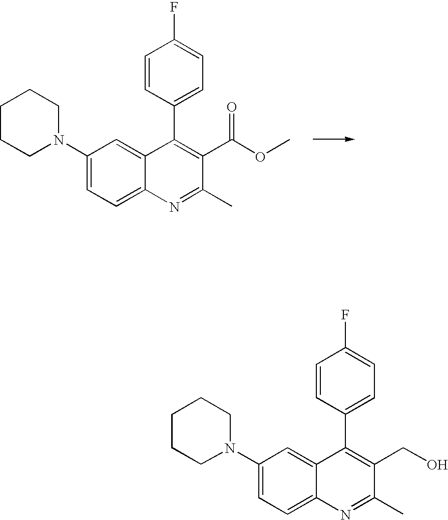

50 mg (0.132 mmol) of methyl 4-(4-fluorophenyl)-2-methyl-6-piperidin-1-ylquinoline-3-carboxylate, ref. step 1q product in Example 25, were dissolved in 5 ml anhydrous THF under a nitrogen atmosphere. The solution was cooled to 0° C. and 0.66 ml (0.66 mmol) of a 1 M LiAlH₄ solution in THF was added dropwise. The mixture was allowed to warm up to rt and was stirred at rt for 2 h. The mixture was cooled to 0° C. and water was slowly added. The organic phase was extracted with DCM and the combined organic layers were dried by filtration through a phase separator. The solvent was evaporated and the crude product purified by HPFC with ethyl acetate as eluent. 38 mg (0.108 mmol, 82%) of [4-(4-fluorophenyl)-2-methyl-6-piperidin-1-ylquinolin-3-yl]methanol was isolated as a light yellow solid.

¹H NMR (400 MHz, CDCl₃) δ 1.48-1.58 (m, 2H), 1.60-1.68 (m, 4H), 2.87 (s, 3H), 3.02-3.08 (m, 4H), 3.69-3.75 (m, 1H), 4.54 (s, 2H), 6.64 (d, 2.7 Hz, 1H), 7.18-7.25 (m, 2H), 7.26-7.32 (m, 2H), 7.44 (dd, 9.3 Hz, 2.7 Hz, 1H), 7.89-8.04 (m, 1H).

MS m/z 351.0 (M+H)⁺.

### Step 1s

### Example 25

### Synthesis of 3-[(4-chlorophenoxy)methyl]-4-(4-fluorophenyl)-2-methyl-6-piperidin-1-ylquinoline

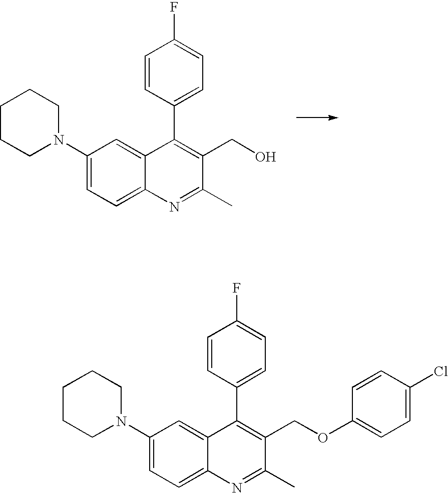

35 mg (0.1 mmol) of [4-(4-fluorophenyl)-2-methyl-6-piperidin-1-ylquinolin-3-yl]methanol, ref. step 1r product in Example 25, were dissolved in 0.5 ml anhydrous THF and 19 mg (0.15 mmol) 4-chlorophenol and 37 mg (0.14 mmol) triphenyl phosphine were added. The mixture was cooled to 0° C. and 0.032 ml (0.15 mmol) diisopropylazodicarboxylate were added dropwise. The mixture was warmed up to rt and stirred at rt for 3 h. The solvent was removed in vacuo and the crude product was purified by reverse phase preparative HPLC. 39 mg (0.085 mmol, 85%) of 3-[(4-chlorophenoxy)methyl]-4-(4-fluorophenyl)-2-methyl-6-piperidin-1-ylquinoline was isolated as light yellow solid.

¹H NMR (400 MHz, CDCl₃) δ 1.49-1.56 (m, 2H), 1.60-1.68 (m, 4H), 2.67 (s, 3H), 3.03-3.07 (m, 4H), 4.77 (s, 2H), 6.55 (d, 2.7 Hz, 1H), 6.75 (d, 8.9 Hz, 2H), 7.10-7.21 (m, 4H), 7.23-7.30 (m, 2H), 7.46 (dd, 9.2 Hz, 2.7 Hz, 1H), 7.92 (d, 9.2 Hz, 1H).

HRMS Calcd for [C₂₈H₂₆ClFN₂O+H]⁺: 461.1796. Found: 461.1782.

### Biological Evaluation

### Effects of the Compounds Acting as GABA_{B} Positive Allosteric Receptor Modulators (PAM) or Agonists in Functional In Vitro Assays.

The effect of GABA in an automated GTPγS³⁵ radioligand filtration-binding assay in CHO cells expressing the GABA_{B(1A,2)} receptor heterodimer was studied in the presence or absence of the positive allosteric modulator test compounds. The positive allosteric modulator according to the invention increased both the potency and the efficacy of GABA.

The potency of the compounds i.e. the ability of the compounds to reduce the EC₅₀ of GABA was revealed by the concentration required to reduce GABA's EC₅₀ by 50%. The potency and efficacy of the compounds acting as agonists at the GABA_{B} receptor was also determined in a automated GTPγS³⁵ radioligand filtration-binding assay.

GTPγS35 Assay principle

The GABA_{B} receptor is a G-protein coupled receptor. Binding of a ligand activates the receptor leading to recruitment of G-protein and a substitution of the G-protein bound GDP to GTP. The G-protein becomes active. The G-protein is inactivated by hydrolysis of GTP to GDP. G-proteins are membrane bound and therefore present in membrane preparations.

In the GTPγS³⁵ assay, GTP is not present but instead GTPγS³⁵ where one of the phosphate groups are substituted to a sulphur group which cannot be hydrolysed. Upon activation of the receptor, radiolabelled GTPγS³⁵ replaces the GDP. The complex cannot be inactivated and the radiolabelled complex is accumulating. At the end of the assay, the reaction mixture is filtered through a membrane-binding filter. Excess GTPγS³⁵ is removed by washing and the membrane bound S³⁵, which correlates to the degree of receptor activation, is measured with a β-Liquid Scintillation Counter.

### Experimental Procedures

### Materials and Reagents

HEPES, GDP, Trizma-HCl, Trizma Base, and Saponin were from Sigma-Aldrich; EDTA, NaCl and MgCl₂×6H₂O were from Merck; Sucrose was from BDH Laboratory supplies; EDTA was from USB Corporation; GABA was from Tocris; GTPγS³⁵ was from Amersham Radiochemicals (GE Healthcare); OptiPhase Supermix was from PerkinElmer; 384 well PS-microplates were from Greiner; 1.2 mL Square well storage plates, low profile were from Abgene; MultiScreen HTS 384 FB (1.0/0.65 μm) filter plates were from Millipore; Biomek AP96 P20 pipette tips (non sterile) were from Beckman; Nut mix F-12 (Ham), DMEM/F12, OptiMEM, penicillin/streptomycin solution (PEST), Lipofectamine, Zeocin, Hygromycin and Geneticin were from Invitrogen; FBS was from Hyclone. Accutase was from Innovative Cell Technologies.

### Generation of Cell Lines Expressing the GABA_{B} Receptor

### Cell Line Used for the Determination of the Test Compounds PAM Potency

GABA_{B}R1a and GABA_{B}R2 were cloned from human brain cDNA and subcloned into pCI-Neo (Promega) and pALTER-1 (Promega), respectively.

In order to optimise the Kozak consensus sequence of GABA_{B}R2, in situ mutagenesis was performed using the Altered Sites Mutagenesis kit according to manufacturer's instruction (Promega) with the following primer, 5′-GAATTCGCACCATGGCTTCCC-3′. The optimised GABA_{B}R2 was then restricted from pALTER-1 with Xho I+Kpn I and subcloned into the mammalian expression vector pcDNA3.1 (−)/Zeo (Invitrogen) to produce the final construct, pcDNA3.1 (−)/Zeo-GABA_{B}R2.

For generation of stable cell lines, CHO-K1 cells were grown in Nut mix F-12 (Ham) media supplemented with 10% FBS, 100 U/ml Penicillin and 100 μg/ml Streptomycin at 37° C. in a humidified CO₂-incubator. The cells were detached with 1 mM EDTA in PBS and 1 million cells were seeded in 100 mm petri dishes. After 24 hours the culture media was replaced with OptiMEM and incubated for 1 hour in a CO₂-incubator.

For generation of a cell line expressing the GABA_{B}R1a/GABA_{B}R2 heterodimer, GABA_{B}R1a plasmid DNA (4 Hg) GABA_{B}R2 plasmid DNA (4 μg) and lipofectamine (24 μl) were mixed in 5 ml OptiMEM and incubated for 45 minutes at room temperature. The cells were exposed to the transfection medium for 5 hours, which then was replaced with culture medium. The cells were cultured for an additional 10 days before selection agents (300 μg/ml hygromycin and 400 μg/ml geneticin) were added. Twenty-four days after transfection, single cell sorting into 96-well plates by flow cytometry was performed using a FACS Vantage SE (Becton Dickinson, Palo Alto, Calif.). After expansion, the GABA_{B} receptor functional response was tested by measuring the GABA_{B} receptor dependent release of intracellular calcium in a fluorescence imaging plate reader (FLIPR). The clone with the highest functional response was collected, expanded and then subcloned by single cell sorting. The clonal cell line with the highest peak response in the FLIPR was used in the present study.

### Cell Line Used for the Determination of the Test Compounds Agonist Potency

The human GABA_{B}R1a was subcloned into pIRESneo3 (Clontech) using GABA_{B}R1a construct as a template (refseqN NM001470). GABA_{B}R2 was subcloned into pcDNA5/FRT (Invitrogen) using GABA_{B}R2 construct as a template (refseqN NM005458). The Kozak sequence GCCACC was introduced before the start codon in both constructs.

For generation of stable cell lines, CHO K1 Flp-In cells (Invitrogen) were grown in DMEM/F12 1:1 media supplemented with 10% FBS at 37° C. in a humidified CO₂-incubator. The cells were detached with Accutase and 1.5 million cells were seeded into T75 flasks. After 24 h, transfection of the cells were performed with the GABA_{B}R2 construct. For generation of cell lines expressing GABA_{B}R2, GABA_{B}R2 plasmid (1 μg) and pOG44 from Invitrogen (9 μg) were mixed with 30 μl Lipofectamine 2000 in 600 μl OptiMEM for 20 minutes. The cells were exposed to transfection medium for 5 hours and was then replaced with culture medium. After 2 days 0.5 mg/ml Hygromycin were added to culture medium. The cells were cultured for an additional 10 days to establish a stable cell mix expressing GABA_{B}R2. For generation of a cell line expressing the GABA_{B}R1a/GABA_{B}R2 hetrodimer, GABA_{B}R1a plasmid DNA (8 μg) and Lipofectamine (30 μl) were mixed in 600 μl OptiMEM and incubated for 20 minutes before added to CHO-Flp-In cells expressing GABA_{B}R2. After 2 days additional selection agent was added (0.8 mg/ml Geneticin). The cells were cultured for another 10 days to generate a stable mixed population expressing the GABA_{B}R1a/GABA_{B}R2 heterodimer. The cell line was analyzed by GTPγS³⁵ assay with GABA as agonist.

### GTPγS³⁵ Assay for Determination of PAM Potency

GTPγS³⁵ radioligand filtration-binding assays were performed using an automated workstation at 30° C. for 1 hour in assay buffer (50 mM HEPES, 40 mM NaCl, 1 mM MgCl₂×6H₂O, 30 μg/mL Saponin, pH 7.4 at RT) containing 0.025 μg/μL of membrane protein (prepared from the cell line described above), 10 μM GDP and 0.55 nCi/μL GTPγS³⁵ in a final volume of 60 μL. The reaction was started by the addition of serially diluted GABA (final start concentration 1 mM dilution factor 3) in the presence or absence of four concentrations (final conc 10, 1, 0.1 and 0.01 μM) of PAM. The reaction was terminated and membranes collected by addition of ice-cold wash buffer (50 mM Tris-HCl, 5 mM MgCl₂×6H₂O, 50 mM NaCl, pH 7.4 at 4° C.) followed by rapid filtration under vacuum through a MultiScreen HTS 384 FB filter plate. Repeated washing of the filters with ice-cold wash buffer washed the unbound radioligand away. The filter plates were dried for 1½-2 hours at 50° C., then 8 μL scintillation liquid was added per well followed by incubation at RT for at least 20 minutes before bound radioactivity was determined using a β-Liquid Scintillation Counter (1450 Microbeta Trilux, Wallac, Finland)

### GTPγS³⁵ Assay for Determination of Agonist Potency

GTPγS³⁵ radioligand filtration-binding assays were performed using an automated workstation at 30° C. for 1 hour in assay buffer (50 mM HEPES, 40 mM NaCl, 1 mM MgCl₂×6H₂O, 30 μg/mL Saponin, pH 7.4 at RT) containing 0.025 μg/μL of membrane protein (prepared from the cell line described above), 10 μM GDP and 0.55 nCi/μL GTPγS³⁵ in a final volume of 60 μL. The reaction was started by the addition of compounds (GABA was always included as a positive control), start concentration 100 μM dilution factor 3. The reaction was terminated and membranes collected by addition of ice-cold wash buffer (50 mM Tris-HCl, 5 mM MgCl₂×6H₂O, 50 mM NaCl, pH 7.4 at 4° C.) followed by rapid filtration under vacuum through a MultiScreen HTS 384 FB filter plate. Repeated washing of the filters with ice-cold wash buffer washed the unbound radioligand away. The filter plates were dried for 1½-2 hours at 50° C., then 8 μL scintillation liquid was added per well followed by incubation at RT for at least 20 minutes before bound radioactivity was determined using a β-Liquid Scintillation Counter (1450 Microbeta Trilux, Wallac, Finland)

### Calculation and Interpretation of Results

### Controls

100% activation (max) is calculated as the mean value for wells containing 1 mM GABA. 0% activation (min) is calculated as the mean value for the wells with DMSO added instead of compound.

### Calculation of Results

All values are calculated as Compound % activation=100*[(X−min)/(max−min)], where X is representing raw value for the compound.

### Test Compound PAM Potency:

EC₅₀, max, min and slope values were calculated from GABA dose-response curves in the presence and absence of PAM constructed using a 4 Parameter Logistic Model (A+((B−A)/(1+((C/x)^{D})))) with XLfit (Model 205, Version 4.2.2, IDBS Solutions), where C=EC₅₀ and D=Slope Factor.

The potency (PAM EC₅₀) of the PAM in GTPγS assays was determined by plotting the log EC₅₀ for GABA against the four log concentrations of the positive allosteric modulator in the presence of which the measurement was performed, using the 4 Parameter Logistic Model described above (slope fixed to 1).

### Test Compound Agonist Potency:

EC₅₀, max, min and slope values were calculated from compound (or GABA) concentration response curves constructed using a 4 Parameter Logistic Model (A+((B−A)/(1+((C/x)^{D})))) with XLfit (Model 205, Version 4.2.2, IDBS Solutions), where C=EC₅₀ and D=Slope Factor.

Generally, the potency of the compounds of formula (I) ranges from EC₅₀s between 40 μM and 0.001 μM. Hereinbelow, individual EC₅₀ values are presented.

| | | Mean (EC₅₀ derived) |
|---|---|---|
| Compound | Mean agonist EC₅₀ (μM) | PAM EC₅₀ (μM) |
| Example 2 | 6.5 μM | 9.2 μM |
| Example 4 | 1.0 μM | 24 μM |
| Example 5 | 1.8 μM | — |
| Example 6 | 2.9 μM | — |
| Example 7 | 1.5 μM | — |
| Example 8 | 1.7 μM | — |
| Example 9 | 3.0 μM | — |
| Example 10 | 1.3 μM | — |
| Example 11 | 1.6 μM | — |
| Example 12 | 2.2 μM | — |
| Example 13 | 0.3 μM | — |
| Example 14 | 4.5 μM | — |
| Example 15 | 0.9 μM | — |
| Example 16 | 24.0 μM | — |
| Example 17 | 7.6 μM | — |
| Example 18 | 4.3 μM | — |
| Example 19 | 1.2 μM | — |
| Example 20 | — | 3.1 μM |
| Example 22 | 1.3 μM | 18 μM |
| Example 25 | 1.1 μM | — |

1. A compound of the formula 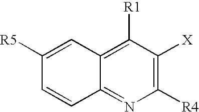
or a pharmaceutically acceptable salt thereof, wherein:
X is —CO—R⁶ or CH(R³)—R²
R¹ is phenyl substituted by one or more halogens;
R² is selected from the group consisting of aryloxy substituted by one or more of C₁-C₁₀-alkyl, C₁-C₁₀-alkoxy, hydroxy, halogen, cyano, C₁-C₁₀-alkylsulfonyl, di-C₁-C₁₀-alkylamino, or carbamoyl; heteroaryloxy; and heteroaryl substituted by one or more of oxo;
R³ is selected from the group consisting of hydrogen and C₁-C₁₀-alkyl;
R⁴ is C₁-C₁₀-alkyl;
R⁵ is selected from the group consisting of halogen and heterocyclyl, unsubstituted or optionally substituted by one or more of C₁-C₁₀-alkyl; and
R⁶ is O—C(R⁷)(R⁸)(R⁹), wherein R⁷, R⁸ and R⁹ are each independently C₁-C₁₀-alkyl, provided that R⁶ is C₁-C₁₀-alkoxy.

2. The compound according to claim 1, wherein
R¹ is 4-fluorophenyl;
R² is selected from the group consisting of phenoxy substituted by one or more of isopropyl, methoxy, hydroxy, chloro, cyano, methanesulfonyl, dimethylamino, or carbamoyl; pyridinyloxy; and 2-pyridin-2(1H)-onyl;
R³ is selected from the group consisting of hydrogen or methyl;
R⁴ is methyl;
R⁵ is selected from the group consisting of bromo, 1-piperidinyl and 4-methyl-1-piperazinyl; and
R⁶ is tert-butoxy.

3. The compound according to claim 1, which is selected from the group consisting of:
6-bromo-3-[1-(4-chlorophenoxy)ethyl]-4-(4-fluorophenyl)-2-methylquinoline;
3-[1-(4-chlorophenoxy)ethyl]-4-(4-fluorophenyl)-2-methyl-6-piperidin-1-ylquinoline;
4-(4-fluorophenyl)-2-methyl-6-piperidin-1-yl-3-[1-(pyridin-2-yloxy)ethyl]quinoline;
4-{1-[4-(4-fluorophenyl)-2-methyl-6-piperidin-1-ylquinolin-3-yl]ethoxy}benzonitrile;
3-{1-[4-(4-fluorophenyl)-2-methyl-6-piperidin-1-ylquinolin-3-yl]ethoxy}benzonitrile;
4-(4-fluorophenyl)-2-methyl-3-{1-[4-(methylsulfonyl)phenoxy]ethyl}-6-piperidin-1-ylquinoline;
4-(4-fluorophenyl)-2-methyl-6-piperidin-1-yl-3-[1-(pyridin-3-yloxy)ethyl]quinoline;
3-[1-(2-chlorophenoxy)ethyl]-4-(4-fluorophenyl)-2-methyl-6-piperidin-1-ylquinoline;
4-(4-fluorophenyl)-3-[1-(4-methoxyphenoxy)ethyl]-2-methyl-6-piperidin-1-ylquinoline;
(3-{1-[4-(4-fluorophenyl)-2-methyl-6-piperidin-1-ylquinolin-3-yl]ethoxy}phenyl)dimethylamine;
4-(4-fluorophenyl)-3-[1-(2-isopropylphenoxy)ethyl]-2-methyl-6-piperidin-1-ylquinoline;
3-{1-[4-(4-fluorophenyl)-2-methyl-6-piperidin-1-ylquinolin-3-yl]ethoxy}benzamide;
2-{1-[4-(4-fluorophenyl)-2-methyl-6-piperidin-1-ylquinolin-3-yl]ethoxy}benzonitrile;
1-{1-[4-(4-fluorophenyl)-2-methyl-6-piperidin-1-ylquinolin-3-yl]ethyl}pyridin-2(1H)-one;
3-[1-(4-chlorophenoxy)ethyl]-4-(4-fluorophenyl)-2-methyl-6-(4-methylpiperazin-1-yl)quinoline;
4-(4-fluorophenyl)-3-[1-(2-isopropylphenoxy)ethyl]-2-methyl-6-(4-methylpiperazin-1-yl)quinoline;
4-{1-[4-(4-fluorophenyl)-2-methyl-6-piperidin-1-ylquinolin-3-yl]ethoxy}phenol;
tert-butyl 6-bromo-4-(4-fluorophenyl)-2-methylquinoline-3-carboxylate;
tert-butyl 4-(4-fluorophenyl)-2-methyl-6-piperidin-1-ylquinoline-3-carboxylate; and
3-[(4-chlorophenoxy)methyl]-4-(4-fluorophenyl)-2-methyl-6-piperidin-1-ylquinoline.

4. (canceled)

5. (canceled)

6. A pharmaceutical composition comprising a compound according to any one of claims 1 to 3 as an active ingredient and a pharmaceutically acceptable carrier or diluent.

7. A method for the treatment or inhibition of gastroesophageal reflux disease (GERD), the method comprising administering a therapeutically effective amount of a compound according to any one of claims 1 to 3, optionally in combination with a GABA_{B} receptor agonist, to a patient in need thereof.

8. A method for the treatment or inhibition of reflux, the method comprising administering a therapeutically effective amount of a compound according to any of claims 1 to 3, optionally in combination with a GABA_{B} receptor agonist, to a patient in need thereof.

9. A method for the treatment or inhibition of transient lower esophageal sphincter relaxations (TLESRs), the method comprising administering a therapeutically effective amount of a compound according to any one of claims 1 to 3, optionally in combination with a GABA_{B} receptor agonist, to a patient in need thereof.

10. A method for the treatment or inhibition of a functional gastrointestinal disorder, the method comprising administering a therapeutically effective amount of a compound according to any one of claims 1 to 3, optionally in combination with a GABA_{B} receptor agonist, to a patient in need thereof.

11. The method according to claim 10, wherein said functional gastrointestinal disorder is functional dyspepsia.

12. A method for the treatment or inhibition of irritable bowel syndrome (IBS), the method comprising administering a therapeutically effective amount of a compound according to claims 1 to 3, optionally in combination with a GABA_{B} receptor agonist, to a patient in need thereof.

13. The method according to claim 12, wherein said IBS is constipation predominant IBS.

14. The method according to claim 12, wherein said IBS is diarrhea predominant IBS.

15. The method according to claim 12, wherein said IBS is alternating bowel movement predominant IBS.
